(19) United States
(12) Patent Application Publication
(10) Pub. No.: US 2010/0068151 A1
(43) Pub. Date: Mar. 18, 2010

(54) MULTIMODALITY MOLECULAR IMAGING WITH THERAPEUTIC CONJUGATES

(76) Inventors: Michael G. Rosenblum, Sugar Land, TX (US); Xiaoyuan Chen, Union City, CA (US)

Correspondence Address: FULBRIGHT & JAWORSKI, L.L.P., 600 CONGRESS AVENUE, SUITE 2400, AUSTIN, TX, 78701, US

(21) Appl. No.: 12/402,301

(22) Filed: Mar. 11, 2009

Related U.S. Application Data
(60) Provisional application No. 61/035,661, filed on Mar. 11, 2008.

(51) Int. Cl. A61K 39/395; C07K 16/00; C07K 14/475; C07K 14/52; A61K 38/48; A61K 49/16; A61K 49/04; A61K 49/22; A61P 35/00

(52) U.S. Cl. 424/9.34; 530/391.7; 530/399; 530/351; 424/178.1; 424/94.65; 424/9.4; 424/9.5

(57) ABSTRACT

Disclosed are pharmaceutical compounds comprising a cell-specific targeting moiety, an anti-cell proliferation moiety, and a chelator moiety. Also disclosed are methods for treating a subject with a hyperproliferative disease, methods for diagnosing presence of a hyperproliferative disease in a subject, and methods for detecting a therapeutic response in a subject that employ the pharmaceutical compounds of the present invention.

The present application claims the benefit of priority to U.S. Provisional Patent Application Ser. No. 61/035,661, filed Mar. 11, 2008, which is hereby incorporated by reference in its entirety.

The government owns rights in the present invention pursuant to grant number R21 EB001785 from the National Institute of Biomedical Imaging and Bioengineering; grant number R24 CA93862 from the National Cancer Institute.

### BACKGROUND OF THE INVENTION

1. Field of the Invention

The present invention relates to the fields of protein chemistry, toxicology, imaging, and the diagnosis and treatment of hyperproliferative disease. More particularly, the invention concerns pharmaceutical compounds that include a cell-specific targeting moiety, an anti-cell proliferation moiety, and a chelator, and therapeutic and diagnostic applications of these compounds.

2. Description of Related Art

Angiogenesis in tumor growth, invasion, and metastasis has been a major focus of recent cancer research, prompting the development of numerous antiangiogenic therapies. Examples of proteins regulating angiogenesis include vascular endothelial growth factor (VEGF) A (VEGF-A), tumor necrosis factor-α, epidermal growth factor, hypoxia-inducible factors, and interleukin 8 (Mohamedali et al., 2005). The receptors for these proteins have been demonstrated in both animal models (Furumatsu et al., 2002) and clinical studies (Bikfalvi and Bicknell, 2002) to be upregulated on tumor vasculature, thus providing therapeutic targets that bypass the tumor parenchyma. Cai et al. conducted studies concerning PET imaging of VEGFR receptor expression in vivo. VEGF-A has been well studied for its prominent role in regulating tumor angiogenesis (Kumamoto et al., 2002), along with its receptors, FLT-1 (VEGFR-1) and KDR (VEGFR-2) in humans and Flt-1 and Flk-1 in mice (Fine et al., 2000). These receptors are expressed in various solid neoplasms, including, but not limited to, bladder (Mohamedali et al., 2005), breast (Price et al., 2001), and pancreatic (Itakura et al., 2000) tumors. Glioblastoma multiforme is a prime example of VEGF-induced tumor neovascularization, as the upregulation of VEGFR-2 on tumor vasculature and the overexpression of VEGF-A ligand by tumor cells have been implicated as poor prognostic markers for gliomas (Fischer et al., 2005).

FLT-1 and KDR are prime targets for antiangiogenic therapies because they are upregulated on tumor vascular endothelium but not on surrounding normal quiescent vessels (Senger et al., 1993). Studies have shown that VEGFR-2 is primarily responsible for the proangiogenic effects of VEGF in solid tumors (Brekken et al., 2000). Tyrosine kinase inhibitors, monoclonal antibodies, ribozymes, small interfering RNA, and soluble receptors that block the VEGF signaling cascade are all currently being developed and tested (Brekken et al., 2000; Fabbro et al., 2000). A novel 84-kDa vasculature-targeting fusion protein (VEGF₁₂₁/rGel) composed of the VEGF-A isoform VEGF₁₂₁ linked with a G₄S tether to recombinant plant toxin gelonin (rGel) has been previously characterized (Veenendaal et al., 2002). The VEGF₁₂₁/rGel fusion toxin has been shown to be highly specific and cytotoxic for both quiescent and dividing porcine aortic endothelial (PAE) cells expressing VEGFR-2 (PAE/KDR) but not for PAE cells expressing VEGFR-1 (PAE/FLT-1) (Veenendaal et al., 2002). VEGF₁₂₁/rGel has also been shown to completely suppress ocular neovascularization (Akiyama et al., 2005) and inhibit the growth of melanomas as well as prostate (Veenendaal et al., 2002), breast (Ran et al., 2005), and bladder (Mohamedali et al., 2005) tumors in mouse models with low systemic toxicity.

There are large variations in the expression of cell surface proteins in solid tumors and receptor modulation in developing tumor neovasculature, and little is known about the precise in vivo fluctuations of receptor-related content (Eggert et al., 2000). Therefore, there is the need for effective noninvasive in vivo imaging techniques for monitoring expression of receptors, such as VEGFR, and evaluating treatment efficacy.

### SUMMARY OF THE INVENTION

The present invention is in part directed to the finding that pharmaceutical compounds that include a chelator conjugated to a cell-specific targeting moiety and an anti-cell proliferation moiety can specifically target and damage hyperproliferative tissue. This targeting and damage can be monitored using various imaging techniques known to those of ordinary skill in the art. For example, in one embodiment it has been found that VEGF₁₂₁/rGel can specifically target and damage tumor vasculature, and this targeting and damage can be monitored using various imaging techniques known to those of ordinary skill in the art. Treatment-induced alterations in the DNA proliferative index can be analyzed using a variety of imaging modalities, such as ¹⁸F-fluorothymidine (¹⁸F-FLT) imaging (Rasey et al., 2002). Tumor growth changes can be monitored by use of imaging modalities known to those of ordinary skill in the art, such as bioluminescence imaging (BLI) and MRI. Thus, the pharmaceutical compounds of the present invention have broad application in combined clinical diagnostic imaging with therapy, allowing for an effective means of monitoring and individualizing the treatment of hyperproliferative disease, such as tumors with neovascularization. Further, the technology of the present invention can be applied in clarifying the mechanisms of resistance to treatment of hyperproliferative disease by other therapies, such as antiangiogenic therapies.

The present invention generally includes pharmaceutical compounds that include one or more cell-specific targeting moieties, one or more anti-cell proliferation moieties, and one or more chelator moieties. The compounds may optionally include a linker moiety linking any of the aforementioned moities to another moiety.

Certain embodiments of the present invention include pharmaceutical compounds comprising an anti-cell proliferation moiety and a cell-specific targeting moiety, wherein the anti-cell proliferation moiety and cell-specific targeting moiety are directly bound to one another or bound to one another by a linker, and one or more chelator moieties is bound to the anti-cell proliferation moiety and/or the cell-specific targeting moiety. Some embodiments of the pharmaceutical compounds include compounds of any of the following formulas:

R₁-(L₁)n₁-R₂-(L₂)n₂-R₃, (I)

R₃-(L₁)n₁-R₁-(L₂)n₂-R₂, (II)

R₂-(L₁)n₁-R₁-(L₂)n₂-R₃, or (III)

R₃-(L₁)n₁-R₂-(L₂)n₂-R₁, (IV)

wherein R₁ is an anti-cell proliferation moiety, R₂ is a cell-specific targeting moiety, R₃ is a chelator moiety, n₁ and n₂ are independent 0 or 1, L₁ is a first linker, and L₂ is a second linker. The compound of any of formulas (I)-(IV) may optionally include more than one additional chelator moities attached to any of R₁, R₂, L₁, and L₂.

Also included in the invention are pharmaceutical compounds of formula:

R₁-(L₁)n₁-R₃-(L₂)n₂-R₂ or (V)

R₂-(L₁)n₁-R₃-(L₂)n₂-R₁, (VI)

wherein R₁ is an anti-cell proliferation moiety that is gelonin, maize RIP, saporin, ricin, ricin A chain, barley RIP, momordin, alpha-momorcharin, beta-momorcharin, Shiga-like RIPE, a-sarcin, TNF-α, Prodigiosin, Pseudomonas exotoxin, Clostridium difficile Toxin B, Helicobacter pylori VacA, Yersinia enterocolitica YopT, Violacein, diethylenetriaminepentaacetic acid, irofulven, Diptheria toxin, mitogillin, ricin, botulinum toxin, cholera toxin, saporin 6, abrin, trichosanthin, dodecandrin, tricokirin, bryodin, or luffin; R₂ is a cell-specific targeting moiety; R₃ is a chelator moiety; n₁ and n₂ are independent 0 or 1; L₁ is a first linker; and L₂ is a second linker. The compounds of the present invention may optionally include one or more additional chelator moieties bound to R₁ and/or R₂.

In some embodiments, there is no linker, and the anti-cell proliferation moiety is directly attached to the cell-specific targeting moiety. In other embodiments, the compound includes a single linker. In further embodiments, the compound includes more than one linker, such as a linker that links a chelator moiety to an anti-cell proliferation moiety or a linker than links a cell-specific targeting moiety to a chelator moiety. Linkers are discussed in greater detail in the specification below.

A cell-specific targeting moiety confers cell-type specific binding to the molecule, and it is chosen on the basis of the particular cell population to be targeted. The cell-specific targeting moiety can be any type of moiety, such as an antibody, a growth factor, a hormone, a polypeptide, a peptide, an aptamer, or a cytokine. In particular embodiments, the cell-specific targeting moiety is an antibody. For example, the antibody may be a full-length antibody, chimeric antibody, Fab′, Fab, F(ab′)₂, single domain antibody (DAB), Fv, single chain Fv (scFv), minibody, diabody, triabody, or a mixture thereof. In a specific embodiment, the antibody is a scFv.

Cell-specific targeting moieties include vascular endothelial cell-specific targeting moieties. Non-limiting examples include VEGF, FGF, integrin, fibronectin, I-CAM, or PDGF. In particular embodiments, the vascular endothelial cell-specific targeting moiety is VEGF. The VEGF may be an isoform such as VEGF₁₂₁, VEGF₁₆₅, VEGF₁₈₉, and VEGF₂₀₆. In a specific embodiment, the isoform is VEGF₁₂₁. The VEGF sequence may be any of SEQ ID NOs:2-8. In other embodiments the cell-specific targeting moiety may be a B lymphocyte stimulator (BLyS) polypeptide. BLyS polypeptides are described, e.g., in U.S. application Ser. No. 11/345,661, which is incorporated herein by reference in its entirety.

Cell-specific targeting moieties also include growth factors. Non-limiting examples of such growth factors include transforming growth factor, epidermal growth factor, insulin-like growth factor, fibroblast growth factor, heregulin, platelet-derived growth factor, vascular endothelial growth factor, and hypoxia inducible factor.

Cell-specific targeting moieties also include hormones. Non-limiting examples include human chorionic gonadotropin, gonadotropin releasing hormone, an androgen, an estrogen, thyroid-stimulating hormone, follicle-stimulating hormone, luteinizing hormone, prolactin, growth hormone, adrenocorticotropic hormone, antidiuretic hormone, oxytocin, thyrotropin-releasing hormone, growth hormone releasing hormone, corticotropin-releasing hormone, somatostatin, dopamine, melatonin, thyroxine, calcitonin, parathyroid hormone, glucocorticoids, mineralocorticoids, adrenaline, noradrenaline, progesterone, insulin, glucagon, amylin, erythropoitin, calcitriol, calciferol, atrial-natriuretic peptide, gastrin, secretin, cholecystokinin, neuropeptide Y, ghrelin, PYY₃₋₃₆, insulin-like growth factor-1, leptin, thrombopoietin, and angiotensinogen.

Cell-specific targeting moieties also include cytokines Non-limiting examples include IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, granulocyte-colony stimulating factor, macrophage-colony stimulating factor, granulocyte-macrophage colony stimulating factor, leukemia inhibitory factor, erythropoietin, granulocyte macrophage colony stimulating factor, oncostatin M, leukemia inhibitory factor, interferon-γ, interferon-α, interferon-β, LT-β, CD40 ligand, Fas ligand, CD27 ligand, CD30 ligand, 4-1BBL, TGFβ, IL-1α, IL-1β, IL-1 RA, MIF, or IGIF.

In some embodiments set forth herein, the compounds include more than one cell-specific targeting moiety or more than one anti-cell proliferation moiety. These moieties can be identical, or distinct.

An “anti-cell proliferation moiety” is defined herein the refer to a molecule or part of a molecule that is capable of reducing or inhibiting the proliferation of a cell. In some embodiments, the anti-cell proliferation moiety is an apoptosis-inducing moiety. An “apoptosis-inducing moiety” is defined herein to refer to any molecule or part of a molecule that is capable of causing a cell to undergo apoptosis. Non-limiting examples of apoptosis-inducing moieties include a granzyme, a Bcl-2 family member, cytochrome C, or a caspase. Non-limiting examples of granzymes include granzyme A, granzyme B, granzyme C, granzyme D, granzyme E, granzyme F, granzyme G, granzyme H, granzyme I, granzyme J, granzyme K, granzyme L, granzyme M, and granzyme N.

Apoptosis-inducing moieties also include Bcl-2 family members. Examples of Bcl-2 family members include Bax, Bak, Bcl-Xs, Bad, Bid, Bik, Hrk, and Bok. The apoptosis-inducing moiety may also be a caspase, such as caspase-1, caspase-2 caspase-3, caspase-4, caspase-5, caspase-6, caspase-7, caspase-8, caspase-9, caspase-10, caspase-11, caspase-12, caspase-13, or caspase-14.

An anti-cell proliferation moiety may also be a cytotoxic agent. A “cytotoxic agent” is defined herein to refer to any agent that is capable of causing injury or death to a cell. The cytotoxic agent may be recombinant.

In certain embodiments, the cytotoxic agent is a ribosome-inhibiting protein (RIP). Non-limiting examples of RIPS include gelonin, maize RIP, saporin, ricin, ricin A chain, barley RIP, momordin, alpha-momorcharin, beta-momorcharin, Shiga-like RIPE, or a-sarcin. In a specific embodiment, the RIP is gelonin. In a more specific embodiment, the RIP is recombinant gelonin.

Other examples of cytotoxic agents include TNF-α, Prodigiosin, Pseudomonas exotoxin, Clostridium difficile Toxin B, Helicobacter pylori VacA, Yersinia enterocolitica YopT, Violacein, diethylenetriaminepentaacetic acid, irofulven, Diptheria toxin, mitogillin, ricin, botulinum toxin, cholera toxin, saporin 6, abrin, trichosanthin, dodecandrin, tricokirin, bryodin, or luffin. In specific embodiments, the cytotoxic agent is TNF-α.

The cell-specific targeting moiety and the anti-cell proliferation moiety may be chemically conjugated or comprised in a fusion polypeptide.

In some embodiments, a linker connects the cell-specific targeting moiety to the anti-cell proliferation moiety. A linker may also connect the chelator moiety to the cell-specific targeting moiety. If more than one linker is present in a single compound, the linkers may be the same or different. Non-limiting examples of linkers include G₄S, (G₄S)₂, (G₄S)₃, 218 linker, an enzymatically cleavable linker, or a pH cleavable linker. In specific embodiments, the compound includes a linker that is G₄S. In the context of the present invention, chelators (discussed below) are not contemplated as linkers. However, a linker may be bound to a chelator.

A “chelator moiety” is defined herein to refer to a molecule that binds to a cation, such as a valent metal ion. Non-limiting examples of chelators include DOTA, DTPA, DMSA, EDTA, Cy-EDTA, EDTMP, DTPA, CyDTPA, Cy2DTPA, BOPTA, DTPA-MA, DTPA-BA, DTPMP, TRITA, TETA, DOTMA, DOTA-MA, HP-DO3A, pNB-DOTA, DOTP, DOTMP, DOTEP, DOTPP, DOTBzP, DOTPME, HEDP, DTTP, an N₃S triamidethiol (MAG3), DADS, MAMA, DADT, a diaminetetrathiol, an N₂P₂ dithiol-bisphosphine, a 6-hydrazinonicotinic acid, a propylene amine oxime, a tetraamine, a cyclal, and a cyclam. In a specific embodiment, the chelator moiety is DOTA. In a further specific embodiment, the chelator is SarAr.

The pharmaceutical compounds of the present invention may include a single chelator moiety, or more than one chelator moiety. If more than one chelator moiety is present, the chelator moieties may be of the same type or distinct. The chelator moiety may be attached to the cell-specific targeting moiety, the anti-cell proliferation moiety, or both the cell-specific targeting moiety and the anti-cell proliferation moiety. In some embodiments, the pharmaceutical compounds include 1 to 20 chelator moieties. In more particular embodiments, the pharmaceutical compounds include 5 to 15 chelator moieties. In a particular embodiment, the pharmaceutical compound includes 8 to 10 chelator moities.

A chelator moiety may be chelated to a valent metal ion. Non-limiting examples of valent metal ions include Cu-64, Cu-60, Cu-61, Cu-62, Cu-67, Lu-177, Zr-89, Y-86, Tc-99m, In-111, Tl-201, Ga-67, Ga-68, As-72, Re-186, Re-188, Ho-166, Y-90, Sm-153, Sr-89, Gd-157, Bi-212, and Bi-213. In a specific embodiment, the valent metal ion is Cu-64.

In a specific embodiment of the pharmaceutical compound, the cell-specific targeting moiety is VEGF₁₂₁, the anti-cell proliferation moiety is recombinant gelonin, and the chelator moiety is DOTA. In a more specific embodiment, Cu-64 is chelated to a chelator moiety.

In a further specific embodiment, the cell-specific targeting moiety is scFvMEL, the anti-cell proliferation moiety is TNF-α, and the chelator moiety is DOTA. In a more specific embodiment, Cu-64 is chelated to a chelator moiety.

In another specific embodiment, the cell-specific targeting moiety is scFv23, the anti-cell proliferation moiety is TNF-α, and the chelator moiety is DOTA. In a more specific embodiment, Cu-64 is chelated to a chelator moiety.

In various embodiments, the cell-specific targeting moiety is BLyS polypeptide or scFvMEL, and the anti-cell proliferation moiety is selected from the group consisting of rGel, TNF-α, GrB, and IKB Inhibitors of NF-kB (IKB) may be used with the present invention and are described, e.g., in U.S. application Ser. No. 11/679,630, which is incorporated herein by reference in its entirety. For example, fusion constructs such as rGel/BLyS, scFvMEL/TNF, scFvMEL/rGel, GrB/scFvMEL, GrB/BLyS, and/or IkB/BLyS may be attached to a chelator moiety and used with the present invention.

The present invention also concerns methods for treating a hyperproliferative disease in a subject, comprising administering to the subject a pharmaceutically effective amount of any of the aforementioned compounds of the present invention.

The subject may be any subject, such as a mammal. Examples include mouse, rat, rabbit, dog, cat, pig, horse, cow, or human. In specific embodiments, the mammal is a human.

A “hyperproliferative disease” is herein defined as any disease associated with abnormal cell growth or abnormal cell turnover. In particular embodiments, the hyperproliferative disease is a disease associated with neovascularization. The disease associated with neovascularization may be cancer. For example, the cancer may be brain cancer, breast cancer, lung cancer, prostate cancer, ovarian cancer, liver cancer, cervical cancer, colon cancer, renal cancer, skin cancer, head and neck cancer, bone cancer, esophageal cancer, bladder cancer, uterine cancer, lymphatic cancer, stomach cancer, pancreatic cancer, testicular cancer, lymphoma, or leukemia. In a particular embodiment, the cancer is brain cancer. In a specific embodiment, the cancer is glioblastoma multiforme.

In some aspects of the present invention, the methods further include treating the subject with one or more additional anti-hyperproliferative therapies. For example, the additional therapy may be chemotherapy, surgery, radiation therapy, gene therapy, hormone therapy, or immunotherapy. The pharmaceutical compound of the present invention may be administered concomitantly with the additional therapy, prior to the additional therapy, or after the additional therapy.

In some embodiments, the method of treatment further comprises imaging the subject using a noninvasive technique, wherein the chelator moiety is chelated to a valent metal ion and the pharmaceutical compound is detectable in vivo using the non-invasive imaging technique. The imaging technique may be any imaging technique known to those of ordinary skill in the art. For example, the imaging technique may be MRI, MR spectroscopy, radiography, CT, ultrasound, planar gamma camera imaging, SPECT, or PET. In a specific embodiment, the imaging technique is PET. Imaging may be performed concomitantly or after administration of the compound.

In some embodiments, the method of treatment is further defined as a method of assessing response to treatment of the hyperproliferative disease.

Other embodiments of the present invention concern methods of diagnosing the presence of a hyperproliferative disease in a subject, involving: (a) administering to a subject a pharmaceutically effective amount of any of the pharmaceutical compounds set forth above, wherein the chelator moiety is chelated to a valent metal; and (b) performing a noninvasive imaging technique, wherein detection of an image identifies the subject as having a hyperproliferative disease. In particular embodiments, the subject is a human. The hyperproliferative disease may be any hyperproliferative disease, but in particular embodiments is a cancer. Examples of cancer are set forth above. In a specific embodiment, the cancer is a brain cancer, such as glioblastoma multiforme. The imaging technique may be any of those imaging techniques discussed above, but in particular embodiments it is PET.

The invention also includes methods for detecting a therapeutic response following administration of a compound of the present invention to a patient with a hyperproliferative disease, comprising the steps of: (1) administering to a subject with a hyperproliferative disease a pharmaceutically effective amount of any of the compounds set forth above, wherein the chelator moiety is chelated to a valent metal ion and is detectable in vivo using a noninvasive imaging technique; (2) imaging the subject using the noninvasive imaging technique to obtain a first image; (3) repeating step (1); and (4) imaging the subject using the noninvasive imaging technique to obtain a second image, wherein reduction of the size or intensity of the second image compared to the first image indicates presence of a therapeutic response. In particular embodiments, the subject is a human, and the disease is cancer. The therapeutic response may be any type of therapeutic response known to those of ordinary skill in the art. In particular embodiments, the therapeutic response is reduction in tumor size or reduction in tumor vascularity.

The present invention also concerns methods for identifying a subject with a tumor that will respond to treatment with a pharmaceutical compound involving a cell-specific targeting moiety conjugated to an anti-cell proliferation moiety, involving the steps of: (1) administering to a subject with a tumor a pharmaceutically effective amount of a pharmaceutical compound as set forth above, wherein the chelator moiety is chelated to a valent metal ion; and (2) performing a noninvasive imaging technique on the subject; wherein presence of a detectable image identifies the subject as having a tumor that will respond to treatment with the compound. In a specific embodiment, the pharmaceutical compound is (64)Cu-DOTA-VEGF(121)/rGel. The tumor may be any type of tumor. Non-limiting examples include a brain cancer, a breast cancer, a lung cancer, a prostate cancer, an ovarian cancer, a liver cancer, a cervical cancer, a colon cancer, a renal cancer, a skin cancer, a head and neck cancer, a bone cancer, an esophageal cancer, a bladder cancer, a uterine cancer, a lymphatic cancer, a stomach cancer, a pancreatic cancer, a testicular cancer, a lymphoma, or a leukemia. In a specific embodiment, the tumor is glioblastoma multiforme.

The imaging technique may be any technique known to those of ordinary skill in the art, including any of those techniques set forth above. In specific embodiments, the imaging is PET with (18)F-FLT.

The present invention also includes kits that include a container and a pharmaceutically effective amount of one or more compounds of the present invention within the container. Kits are discussed in greater detail in the specification below.

The present invention also includes methods of synthesizing the compounds of the present invention, as discussed in greater detail below.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

The use of the term “or” in the claims is used to mean “and/or” unless explicitly indicated to refer to alternatives only or the alternative are mutually exclusive, although the disclosure supports a definition that refers to only alternatives and “and/or.”

Throughout this application, the term “about” is used to indicate that a value includes the standard deviation of error for the device and/or method being employed to determine the value.

As used herein the specification, “a” or “an” may mean one or more, unless clearly indicated otherwise. As used herein in the claim(s), when used in conjunction with the word “comprising,” the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The following figures form part of the present specification and are included to further demonstrate certain aspects of the present invention. The invention may be better understood by reference to one or more of these drawings in combination with the detailed description of specific embodiments presented herein.

FIGS. 1A, 1B. 1A—Cell-binding assay revealed IC₅₀ values of 24.5 and 40.6 nM for VEGF₁₂₁/rGel and DOTA-VEGF₁₂₁/rGel, respectively, demonstrating no significant difference in inhibition of ¹²⁵I-VEGF₁₆₅ binding to PAE/KDR cells after DOTA conjugation. 1B—Functional assay revealed no significant difference in levels of phosphorylated KDR induced by serial concentrations of VEGF₁₂₁/rGel and DOTA-VEGF₁₂₁/rGel.

FIGS. 2A, 2B. 2A—Representative sagittal PET images of ⁶⁴Cu-DOTA-VEGF₁₂₁/rGel tumor accumulation from 1 h to 48 h after injection in glioblastoma tumor-bearing mouse (control). Tumor accumulation steadily increased and peaked at 11.8±2.3% ID/g (n=3) at 18 h after injection. Sagittal PET images of tumor-bearing mouse injected with 200 μg of VEGF₁₂₁ before ⁶⁴Cu-DOTA-VEGF₁₂₁/rGel injection are also shown (block). Yellow arrows indicate tumor location. 2B—Blocking experiment with 200 μg of VEGF₁₂₁ injected before ⁶⁴Cu-DOTA-VEGF₁₂₁/rGel injection revealed significant reduction in ⁶⁴Cu-DOTA-VEGF₁₂₁/rGel tumor uptake. *P<0.05; **P<0.01.

FIGS. 3A, 3B, 3C, 3D. 3A—MRI revealed no difference in tumor volume between groups (control: n=6; treatment: n=9) at baseline and decrease in treatment group tumor volume after 2 doses of VEGF₁₂₁/rGel. Yellow arrows indicate tumor location. 3B—From baseline to after 2 doses of VEGF₁₂₁/rGel, control group tumor volume increased from 2.8±0.8 mm³ to 6.3±2.0 mm³, whereas treatment group tumor volume decreased 1.3-fold, from 3.9±0.7 mm³ to 2.9±0.6 mm³. *P<0.05. 3C—BLI of control and treatment groups revealed no difference in tumor signal intensity at baseline and significant difference after 2 doses of VEGF₁₂₁/rGel. 3D—Control group BLI tumor signal intensity increased from 2.9×10⁷±1.6×10⁷ photons per second to 7.0×10⁷±2.3×10⁷ photons per second from baseline to after 2 doses of VEGF₁₂₁/rGel, whereas treatment group BLI tumor signal intensity increased only slightly, from 2.7×10⁷±1.3×10⁷ photons per second to 3.7×10⁷±1.2×10⁷ photons per second. *P<0.05.

FIG. 4. BLI tumor signal intensities for control and treatment groups from baseline to after 4 doses of VEGF₁₂₁/rGel revealed a peak 8.7-fold decrease in BLI tumor signal intensity in treated mice. *P<0.05.

FIGS. 5A, 5B, 5C. 5A—Representative coronal and sagittal images of ¹⁸F-FLT at 1 h after injection for mouse before and after 4 doses of VEGF₁₂₁/rGel treatment. Yellow arrows indicate tumor location. 5B—Tumor accumulation values at 1 h after injection (n=3) were 2.9±0.7% ID/g for control tumors and 1.7±0.4% ID/g for tumors after 4 doses of VEGF₁₂₁/rGel. Quantification data from ¹⁸F-FLT PET scan at 2 h after injection are also shown. 5C—T/B ratios at 1 h after injection were 4.1±0.7 and 2.3±0.5 for control and treated mice, respectively (n=3). *P<0.05. Data from ¹⁸F-FLT PET scan at 2 h after injection are similar to those at 1 h after injection.

FIGS. 6A, 6B. 6A—Ex vivo histologic and immunohistochemical analyses with H&E, Ki67, and TUNEL at ×20 magnification revealed clear differences between control tumors and tumors after 4 doses of VEGF₁₂₁/rGel. H&E analysis revealed VEGF₁₂₁/rGel-induced damage of tumor vasculature accompanied by inflammation and red blood cell extravasation. Ki67 analysis revealed significant decreases in DNA synthesis and proliferative index in treated tumors compared with control tumors (brown cells are Ki67-positive cells). Quantitative TUNEL analysis demonstrated marked increases in DNA fragmentation and apoptosis in treated mice compared with control mice (green cells are TUNEL-positive cells). 6B—Immunofluorescence analysis with DAPI (blue), CD31 (green), and TUNEL (red) at ×10 magnification revealed significant increases in apoptosis on tumor vasculature and surrounding cells in treated mice compared with control mice, indicating specific VEGF₁₂₁/rGel-induced tumor vasculature damage.

### DESCRIPTION OF ILLUSTRATIVE EMBODIMENTS

The present invention generally concerns the finding that pharmaceutical compounds which include a cell-specific targeting moiety conjugated to an anti-cell proliferation moiety and a chelator have broad application in combined clinical diagnostic imaging with therapy, allowing for an effective means of monitoring and individualizing the treatment of hyperproliferative disease, such as tumors with neovascularization. Further, the technology of the present invention can be applied in clarifying the mechanisms of resistance to treatment of hyperproliferative disease by other therapies.

### A. POLYPEPTIDES

### 1. Polypeptides in General

The present invention concerns pharmaceutical compounds that include a cell-specific targeting moiety conjugated to an anti-cell proliferation moiety. In specific embodiments of the present invention, the cell-specific targeting moiety and the anti-cell proliferation moiety are both polypeptides. As used herein, a “polypeptide” generally is defined herein to refer to a peptide sequence of about 3 to about 10,000 or more amino acid residues.

The term “amino acid” not only encompasses the 20 common amino acids in naturally synthesized proteins, but also includes any modified, unusual, or synthetic amino acid. One of ordinary skill in the art would be familiar with modified, unusual, or synthetic amino acids. Examples of modified and unusual amino acids are shown on Table 1 below.

TABLE 1
Modified and Unusual Amino Acids

| Abbr. | Amino Acid |
|---|---|
| Aad | 2-Aminoadipic acid |
| Baad | 3-Aminoadipic acid |
| Bala | β-alanine, β-Amino-propionic acid |
| Abu | 2-Aminobutyric acid |
| 4Abu | 4-Aminobutyric acid, piperidinic acid |
| Acp | 6-Aminocaproic acid |
| Ahe | 2-Aminoheptanoic acid |
| Aib | 2-Aminoisobutyric acid |
| Baib | 3-Aminoisobutyric acid |
| Apm | 2-Aminopimelic acid |
| Dbu | 2,4-Diaminobutyric acid |
| Des | Desmosine |
| Dpm | 2,2′-Diaminopimelic acid |
| Dpr | 2,3-Diaminopropionic acid |
| EtGly | N-Ethylglycine |
| EtAsn | N-Ethylasparagine |
| Hyl | Hydroxylysine |
| AHyl | allo-Hydroxylysine |
| 3Hyp | 3-Hydroxyproline |
| 4Hyp | 4-Hydroxyproline |
| Ide | Isodesmosine |
| AIle | allo-Isoleucine |
| MeGly | N-Methylglycine, sarcosine |
| MeIle | N-Methylisoleucine |
| MeLys | 6-N-Methyllysine |
| MeVal | N-Methylvaline |
| Nva | Norvaline |
| Nle | Norleucine |
| Orn | Ornithine |

In some embodiments, the polypeptides that are set forth herein are chimeric, in that they comprise a cell-specific targeting moiety and an anti-cell proliferation moiety which are both amino acid sequences. The polypeptides set forth herein may comprise one or more cell-specific targeting moiety amino acid sequences, which may or may not be identical. Similarly, the polypeptides set forth herein may comprise one or more anti-cell proliferation amino acid sequences, which may or may not be identical.

In certain embodiments of the present invention, the polypeptide is a fusion polypeptide that includes cell-specific targeting amino acid sequence linked at the N- or C-terminus of the cell-specific targeting amino acid sequence to a anti-cell proliferation amino acid sequence. In other embodiments, the polypeptide comprises a linker interposed between the two amino acid sequences. Linkers are discussed in greater detail in the specification below.

Furthermore, the polypeptides set forth herein may comprises a sequence of any number of additional amino acid residues at either the N-terminus or C-terminus of the amino acid sequence that includes the cell-specific targeting targeting amino acid sequence and the anti-cell proliferation amino acid sequence. For example, there may be an amino acid sequence of about 3 to about 10,000 or more amino acid residues at either the N-terminus, the C-terminus, or both the N-terminus and C-terminus of the chimeric polypeptide.

The polypeptide may include the addition of an immunologically active domain, such as an antibody epitope or other tag, to facilitate targeting or purification of the polypeptide. The use of 6×His and GST (glutathione S transferase) as tags is well known. Inclusion of a cleavage site at or near the fusion junction will facilitate removal of the extraneous polypeptide after purification. Other amino acid sequences that may be included in the polypeptide include functional domains, such as active sites from enzymes such as a hydrolase, glycosylation domains, cellular targeting signals or transmembrane regions. The polypeptide may further include one or more additional anti-cell proliferation moieties, which are discussed in greater detail in the specification below.

Cell-specific targeting amino acid sequences and anti-cell proliferation amino acid sequences set forth herein may possess deletions and/or substitutions of amino acids relative to the native sequence; thus, sequences with a deletion, sequences with a substitution, and sequences with a deletion and a substitution are contemplated for inclusion in the polypeptides of the present invention. In some embodiments, these targeted polypeptides may further include insertions or added amino acids, such as linkers.

Substitutional or replacement variants typically contain the exchange of one amino acid for another at one or more sites within the protein and may be designed to modulate one or more properties of the polypeptide, particularly to increase its efficacy or specificity. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

In addition to a deletion or substitution, vascular endothelial targeting amino acid sequences and cytotoxic amino acid sequences that are included in the polypeptides set forth herein may possess an insertion one or more residues. This may include the addition of one or more amino acid residues.

The term “biologically functional equivalent” is well understood in the art and is further defined in detail herein. Accordingly, sequences that have between about 70% and about 80%, or between about 81% and about 90%, or even between about 91% and about 99% of amino acids that are identical or functionally equivalent to the amino acids of the native vascular endothelial targeting sequence or cytotic amino acid sequence are included, provided the biological activity of the native sequence is maintained.

Thus, the anti-cell proliferation targeting amino acid sequences and the cell-specific targeting amino acid sequences may be biologically functionally equivalent to the native counterparts. For example, a vascular endothelial targeting amino acid sequence may be functionally equivalent in terms of ability to bind or attach to a vascular endothelial cell. In some embodiments, the cell-specific targeting amino acid sequence or the anti-cell proliferation amino acid sequence has greater biological activity than the native counterpart.

The following is a discussion based upon changing of the amino acids of a polypeptide to create an equivalent, or even an improved, second-generation molecule. For example, certain amino acids may be substituted for other amino acids in a polypeptide without appreciable loss of function, such as ability to interact with an endothelial cell of a blood vessel. Since it is the interactive capacity and nature of a polypeptide that defines that polypeptide's biological functional activity, certain amino acid substitutions can be made in a polypeptide sequence and nevertheless produce a polypeptide with like properties.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still produce a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

### 2. Methods of Polypeptide Synthesis

In certain embodiments of the present invention, a fusion polypeptide is encoded by a single recombinant nucleic acid sequence using recombinant techniques. In other embodiments, the cell-specific targeting amino acid sequence and the anti-cell proliferation amino acid sequence have been encoded by separate nucleic acid sequences, and subsequently joined by chemical conjugation. In further embodiments, the polypeptide has been synthesized de novo.

a. Recombinant Techniques

In certain embodiments of the present invention, a chimeric polypeptide is encoded by a single recombinant polynucleotide using recombinant techniques well-known to those of ordinary skill in the art. The polynucleotide may include a sequence of additional nucleic acids that direct the expression of the chimeric polypeptide in appropriate host cells.

Due to the inherent degeneracy of the genetic code, other DNA sequences that encode substantially the same or a functionally equivalent amino acid sequence, may be used in the practice of the invention of the cloning and expression of the chimeric protein. Such DNA sequences include those capable of hybridizing to the chimeric sequences or their complementary sequences under stringent conditions. In one embodiment, the phrase “stringent conditions” as used herein refers to those hybridizing conditions that (1) employ low ionic strength and high temperature for washing, for example, 0.015 M NaCl/0.0015 M sodium citrate/0.1% SDS at 50° C.; (2) employ during hybridization a denaturing agent such as formamide, for example, 50% (vol/vol) formamide with a 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C.; or (3) employ 50% formamide, 5×SSC (0.75 M NaCl, 0.075 M Sodium pyrophosphate, 5×Denhardt's solution, sonicated salmon sperm DNA (50 μg/ml), 0.1% SDS, and 10% dextran sulfate at 42° C., with washes at 42° C. in 0.2×SSC and 0.1% SDS.

Altered DNA sequences that may be used in accordance with the invention include deletions, additions or substitutions of different nucleotide residues resulting in a sequence that encodes the same or a functionally equivalent polynucleotide. The polynucleotide may contain deletions, additions or substitutions of amino acid residues within a chimeric sequence, which result in a silent change thus producing a functionally equivalent chimeric polynucleotide. Such amino acid substitutions may be made on the basis of similarity in polarity, charge, solubility, hydrophobicity, hydrophilicity, and/or the amphipathic nature of the residues involved, as discussed above.

The DNA sequences of the invention may be engineered in order to alter a chimeric coding sequence for a variety of ends, including but not limited to, alterations that modify processing and expression of the gene product. For example, mutations may be introduced using techniques that are well known in the art, e.g., site-directed mutagenesis, to insert new restriction sites, to alter glycosylation patterns, phosphorylation, etc.

In order to express a biologically active chimeric polypeptide, the nucleotide sequence coding for a chimeric polypeptide, or a functional equivalent, is inserted into an appropriate expression vector, i.e., a vector that contains the necessary elements for the transcription and translation of the inserted coding sequence. The chimeric gene products as well as host cells or cell lines transfected or transformed with recombinant chimeric expression vectors can be used for a variety of purposes. These include, but are not limited to, generating antibodies (i.e., monoclonal or polyclonal) that bind to epitopes of the proteins to facilitate their purification.

Methods that are well known to those skilled in the art can be used to construct expression vectors containing the chimeric coding sequence and appropriate transcriptional/translational control signals. These methods include in vitro recombinant DNA techniques, synthetic techniques and in vivo recombination/genetic recombination. See, for example, the techniques described in Sambrook et al., 2001.

A variety of host-expression vector systems may be utilized to express the chimeric polypeptide coding sequence. These include but are not limited to microorganisms such as bacteria transformed with recombinant bacteriophage DNA, plasmid DNA or cosmid DNA expression vectors containing the chimeric protein coding sequence; yeast transformed with recombinant yeast expression vectors containing the chimeric protein coding sequence; insect cell systems infected with recombinant virus expression vectors (e.g., baculovirus) containing the chimeric protein coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the chimeric protein coding sequence; or animal cell systems. It should be noted that since most apoptosis-inducing proteins cause programmed cell death in mammalian cells, it is preferred that the chimeric protein of the invention be expressed in prokaryotic or lower eukaryotic cells. Section 6 illustrates that IL2-Bax may be efficiently expressed in E. coli.

The expression elements of each system vary in their strength and specificities. Depending on the host/vector system utilized, any of a number of suitable transcription and translation elements, including constitutive and inducible promoters, may be used in the expression vector.

Specific initiation signals may also be required for efficient translation of the inserted chimeric protein coding sequence. These signals include the ATG initiation codon and adjacent sequences. In cases where the entire chimeric gene, including its own initiation codon and adjacent sequences, is inserted into the appropriate expression vector, no additional translational control signals may be needed. However, in cases where the chimeric protein coding sequence does not include its own initiation codon, exogenous translational control signals, including the ATG initiation codon, must be provided. Furthermore, the initiation codon must be in phase with the reading frame of the chimeric protein coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. The presence of consensus N-glycosylation sites in a chimeric protein may require proper modification for optimal chimeric protein function. Different host cells have characteristic and specific mechanisms for the post-translational processing and modification of proteins. Appropriate cell lines or host systems can be chosen to ensure the correct modification and processing of the chimeric protein. To this end, eukaryotic host cells which possess the cellular machinery for proper processing of the primary transcript, glycosylation, and phosphorylation of the chimeric protein may be used. Such mammalian host cells include but are not limited to CHO, VERO, BHK, HeLa, COS, MDCK, 293, W138, and the like.

For long-term, high-yield production of recombinant chimeric polypeptides, stable expression is preferred. For example, cell lines that stably express the chimeric polypeptide may be engineered. Rather than using expression vectors that contain viral originals of replication, host cells can be transformed with a chimeric coding sequence controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines.

b. De Novo Synthesis

In an alternate embodiment of the invention, the chimeric polypeptide could be synthesized de novo in whole or in part, using chemical methods well known in the art (see, for example, Caruthers et al., 1980; Crea and Horn, 1980; and Chow and Kempe, 1981). For example, the component amino acid sequences can be synthesized by solid phase techniques, cleaved from the resin, and purified by preparative high performance liquid chromatography followed by chemical linkage to form a chimeric protein. (e.g., see Creighton, 1983). The composition of the synthetic peptides may be confirmed by amino acid analysis or sequencing (e.g., the Edman degradation procedure; see Creighton, 1983).

Polypeptide synthesis techniques are well known to those of skill in the art (see, e.g., Bodanszky et al., 1976). These synthetic methods involve the sequential addition of one or more amino acid residues or suitable protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group, such as lysine.

Using solid phase synthesis as an example, the protected or derivatized amino acid is attached to an inert solid support through its unprotected carboxyl or amino group. The protecting group of the amino or carboxyl group is then selectively removed and the next amino acid in the sequence having the complementary (amino or carboxyl) group suitably protected is admixed and reacted with the residue already attached to the solid support. The protecting group of the amino or carboxyl group is then removed from this newly added amino acid residue, and the next amino acid (suitably protected) is then added, and so forth. After all the desired amino acids have been linked in the proper sequence, any remaining terminal and side group protecting groups (and solid support) are removed sequentially or concurrently, to provide the final peptide. Such protecting group moieties may be used in the course of synthesis, but they are removed before the peptides are used. Additional reactions may be necessary, as described elsewhere, to form intramolecular linkages to restrain conformation.

The chimeric proteins of the invention can be purified by art-suitable techniques such as high performance liquid chromatography, ion exchange chromatography, gel electrophoresis, affinity chromatography, and the like. The actual conditions used to purify a particular protein will depend, in part, on factors such as net charge, hydrophobicity, hydrophilicity, etc., and will be apparent to those having skill in the art.

Additional information regarding preparation of constructs that include a cell-specific targeting moiety and an anti-cell proliferation moiety can be found in U.S. Patent App. Pub. No. 20050037967, U.S. Pat. No. 6,146,850, U.S. Pat. No. 5,837,491, U.S. Pat. No. 5,744,580, U.S. Pat. No. 5,756,699, U.S. Pat. No. 6,146,631, U.S. RE 37,462, U.S. Pat. No. 5,624,827, and U.S. Patent App. Pub. No. 20070025957, each of which is herein specifically incorporated by reference.

### 3. Protein Purification

In certain embodiments of the present invention, the polypeptide has been purified. Generally, “purified” will refer to a polypeptide composition that has been subjected to fractionation to remove various other components, and which composition substantially retains its expressed biological activity. Where the term “substantially purified” is used, this designation will refer to a composition in which the polypeptide or peptide forms the major component of the composition, such as constituting about 50% to about 99.9% or more of the proteins in the composition.

Various methods for quantifying the degree of purification of the polypeptide will be known to those of skill in the art in light of the present disclosure. Exemplary techniques include high performance liquid chromatography, ion exchange chromatography, gel electrophoresis, affinity chromatography and the like. The actual conditions used to purify a particular polypeptide will depend, in part, on factors such as net charge, hydrophobicity, hydrophilicity, etc., and will be apparent to those having skill in the art.

For affinity chromatography purification, any antibody that specifically binds the polypeptide may be used. For the production of antibodies, various host animals, including but not limited to rabbits, mice, rats, etc., may be immunized by injection with a chimeric protein or a fragment thereof. The protein may be attached to a suitable carrier, such as bovine serum albumin (BSA), by means of a side chain functional group or linkers attached to a side chain functional group. Various adjuvants may be used to increase the immunological response, depending on the host species, including but not limited to, Freund's (complete and incomplete), mineral gels such as aluminum hydroxide, surface active substances such as lysolecithin, pluronic polyols, polyanions, peptides, oil emulsions, keyhold limpet hemocyanin, dinitrophenol, and potentially useful human adjuvants such as BCG (bacilli Calmetter-Guerin) and Corynebacterium parvum.

For affinity chromatography purification, any antibody that specifically binds the protein may be used. For the production of antibodies, various host animals, including but not limited to rabbits, mice, rats, etc., may be immunized by injection with a chimeric protein or a fragment thereof. The protein may be attached to a suitable carrier, such as bovine serum albumin (BSA), by means of a side chain functional group or linkers attached to a side chain functional group. Various adjuvants may be used to increase the immunological response, depending on the host species, including but not limited to, Freund's (complete and incomplete), mineral gels such as aluminum hydroxide, surface active substances such as lysolecithin, pluronic polyols, polyanions, peptides, oil emulsions, keyhold limpet hemocyanin, dinitrophenol, and potentially useful human adjuvants such as BCG (bacilli Calmetter-Guerin) and Corynebacterium parvum.

### B. CELL-SPECIFIC TARGETING MOIETIES

The compounds of the invention are comprised of a cell-specific targeting moiety and an anti-cell proliferation moiety. The cell-specific targeting moiety confers cell-type specific binding to the molecule, and it is chosen on the basis of the particular cell population to be targeted. A wide variety of proteins are suitable for use as cell-specific targeting moieties, including but not limited to, ligands for receptors such as growth factors, hormones and cytokines, and antibodies or antigen-binding fragments thereof. Examples of cell-specific targeting moieties are set forth below.

### 1. Antibodies

In some embodiments of the invention, one or more antibodies are employed as a cell-specific targeting moiety. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

Antibodies are extremely versatile and useful cell-specific targeting moieties because they can be generated against any cell surface antigen of interest. Monoclonal antibodies have been generated against cell surface receptors, tumor-associated antigens, and leukocyte lineage-specific markers such as CD antigens. Antibody variable region genes can be readily isolated from hybridoma cells by methods well known in the art.

Over the past few years, several monoclonal antibodies have been approved for therapeutic use and have achieved significant clinical and commercial success. Much of the clinical utility of monoclonal antibodies results from the affinity and specificity with which they bind to their targets, as well as long circulating life due to their relatively large size. Monoclonal antibodies, however, are not well suited for use in indications where a short half-life is advantageous or where their large size inhibits them physically from reaching the area of potential therapeutic activity.

Moreover, antibodies in their native form, consisting of two different polypeptide chains that need to be generated in approximately equal amounts and assembled correctly, are not optimal candidates for therapeutic purposes. However, it is possible to create a single polypeptide that can retain the antigen binding properties of a monoclonal antibody.

Single chain antibodies (SCAs) are genetically engineered proteins designed to expand on the therapeutic and diagnostic applications possible with monoclonal antibodies. SCAs have the binding specificity and affinity of monoclonal antibodies and, in their native form, are about one-fifth to one-sixth of the size of a monoclonal antibody, typically giving them very short half-lives. Human SCAs offer many benefits compared to most monoclonal antibodies, including more specific localization to target sites in the body, faster clearance from the body, and a better opportunity to be used orally, intranasally, transdermally or by inhalation, for example. In addition to these benefits, fully-human SCAs can be isolated directly from human SCA libraries without the need for costly and time consuming “humanization” procedures. SCAs are also readily produced through intracellular expression (inside cells) allowing for their use in gene therapy applications where SCA molecules act as specific inhibitors of cell function.

Single-chain recombinant antibodies (scFvs) consist of the antibody VL and VH domains linked by a designed flexible peptide tether (Atwell et al., 1999). Compared to intact IfGs, scFvs have the advantages of smaller size and structural simplicity with comparable antigen-binding affinities, and they can be more stable than the analogous 2-chain Fab fragments (Colcher et al., 1998; Adams and Schier, 1999). Several studies have shown that the smaller size of scFvs provides better penetration into tumor tissue, improved pharmacokinetics, and a reduction in the immunogenicity observed with i.v. administered Fabs compared to that of intact murine antibodies (Bird et al., 1988; Colcher et al., 1990; Colcher et al., 1998; Adams and Schier, 1999). For example, the scFvMEL single-chain antibody retains the same binding affinity and specificity of the parental ZME-018 antibody that recognizes the surface domain of the gp240 antigen present on human melanoma cells (Kantor et al., 1982; Macey et al., 1998).

Recombinant single-chain Fv antibody (scFv)-based agents have been used in pre-clinical studies for cell-targeted delivery of cytokines (Liu et al., 2004) and intracellular delivery of highly cytotoxic n-glycosidases such as recombinant gelonin (rGel) toxin (Rosenblum et al., 2003). The smaller size of these antibody fragments may allow better penetration into tumor tissue, improved pharmacokinetics, and a reduction in the immunogenicity observed with intravenously administered murine antibodies. Initially, to target melanoma cells, a recombinant single-chain antibody designated scFvMEL which recognizes the high-molecular-weight glycoprotein gp240, found on a majority (80%) of melanoma cell lines and fresh tumor samples (Kantor et al., 1982) may be used. It has been used extensively to target gp240 bearing cells in vitro and using xenograft models (Rosenblum et al., 2003; Liu et al., 2003; Rosenblum et al., 1991; Rosenblum et al. 1994; Rosenblum et al., 1995; Rosenblum et al., 1996; Rosenblum et al., 1999). This antibody binds to target cells and is efficiently internalized making this an excellent carrier to deliver toxins or other therapeutic payloads.

Antibodies designated ZME-018 or 225.28 S that is the parental antibody of scFvMEL targeting the gp240 antigen have been extensively studied in melanoma patients and have demonstrated an impressive ability to localize in metastatic tumors after systemic administration (Rosenblum et al., 1994; Kantor et al., 1986; Macey et al., 1988; Rosenblum et al., 1991). This antibody possesses high specificity for melanoma and is minimally reactive with a variety of normal tissues, making it a promising candidate for further study (Rosenblum et al., 1995; Macey et al., 1988; Rosenblum et al., 1991; Mujoo et al., 1995). More importantly, the gp240 antigen is not expressed on normal cells thus making this an interesting target for therapeutic intervention.

The variable regions from the heavy and light chains (VH and VL) are both approximately 110 amino acids long. They can be linked by a 15 amino acid linker with the sequence (SEQ ID NO:9), for example, which has sufficient flexibility to allow the two domains to assemble a functional antigen binding pocket. In specific embodiments, addition of various signal sequences allows the scFv to be targeted to different organelles within the cell, or to be secreted. Addition of the light chain constant region (Ck) allows dimerization via disulfide bonds, giving increased stability and avidity. Thus, for a single chain Fv (scFv) SCA, although the two domains of the Fv fragment are coded for by separate genes, it has been proven possible to make a synthetic linker that enables them to be made as a single protein chain scFv (Bird et al., 1988; Huston et al., 1988) by recombinant methods. Furthermore, they are frequently used due to their ease of isolation from phage display libraries and their ability to recognize conserved antigens (for review, see Adams and Schier, 1999). For example, scFv is utilized to target suicide genes to carcinoembryonic antigen (CEA)-expressing tumor cells by a retrovector displaying anti-CEA scFv (Kuroki et al., 2000).

Furthermore, the Fc portion of the heavy chain of an antibody may be used to target Fc receptor-expressing cells such as the use of the Fc portion of an IgE antibody to target mast cells and basophils. The use of antibodies to target a polypeptide or peptide of interest by antibody-directed therapy or immunological-directed therapy is currently approved and in use in the present therapeutic market.

A Fab antibody fragment may be utilized in the invention. An Fab fragment comprises a light chain and the N-terminal portion of the heavy chain that are linked together by disulfide bonds. It typically has a molecular weight of approximately 50 kD and comprises a single antigen binding site. Fab fragments may be obtained from F(ab′).sub.2 fragments by limited reduction, or from whole antibody by digestion with papain in the presence of reducing agents.

### 2. Moieties Other than Antibodies

Molecules other than antibodies or antibody fragments may be employed as cell-specific targeting moieties. Since a large number of cell surface receptors have been identified in hematopoietic cells of various lineages, ligands or antibodies specific for these receptors may be used as cell-specific targeting moieties. IL2 may be used as a cell-specific targeting moiety in a chimeric protein to target IL2R+ cells. Alternatively, other molecules such as B7-1, B7-2 and CD40 may be used to specifically target activated T cells (Barclay et al., 1993). Furthermore, B cells express CD19, CD40 and IL4 receptor and may be targeted by moieties that bind these receptors, such as CD40 ligand, IL4, IL5, IL6 and CD28. The elimination of immune cells such as T cells and B cells is particularly useful in the treatment of autoimmunity, hypersensitivity, transplantation rejection responses and in the treatment of lymphoid tumors. Examples of autoimmune diseases are multiple sclerosis, rheumatoid arthritis, insulin-dependent diabetes mellitus, systemic lupus erythemotisis, scleroderma, and uviatis. More specifically, since myelin basic protein is known to be the major target of immune cell attack in multiple sclerosis, this protein may be used as a cell-specific targeting moiety for the treatment of multiple sclerosis (WO 97/19179).

Other cytokines that may be used to target specific cell subsets include the interleukins (IL1 through IL15), granulocyte-colony stimulating factor, macrophage-colony stimulating factor, granulocyte-macrophage colony stimulating factor, leukemia inhibitory factor, tumor necrosis factor, transforming growth factor, epidermal growth factor, insulin-like growth factors, and/or fibroblast growth factor (Thompson, 1994).

A skilled artisan recognizes that there are a variety of known cytokines, including hematopoietins (four-helix bundles) (such as Epo (erythropoietin), IL-2 (T-cell growth factor), IL-3 (multicolony CSF), IL-4 (BCGF-1, BSF-1), IL-5 (BCGF-2), IL-6 IL-4 (IFN-.beta.2, BSF-2, BCDF), IL-7, IL-8, IL-9, IL-11, IL-13 (P600), G-CSF, IL-15 (T-cell growth factor), GM-CSF (granulocyte macrophage colony stimulating factor), OSM [OM, oncostatin M), and LIF (leukemia inhibitory factor)]; interferons (such as IFN-.gamma., IFN-α, and IFN-β; immunoglobin superfamily (such as B7.1 (CD80), and B7.2 (B70, CD86)); TNF family (such as TNF-α (cachectin), TNF-β (lymphotoxin, LT, LT-α), LT-β, CD40 ligand (CD40L), Fas ligand (FasL), CD27 ligand (CD27L), CD30 ligand (CD30L), and 4-1BBL)); and those unassigned to a particular family [such as TGF-β, IL 1-α, IL-1β, IL-1 RA, IL-10 (cytokine synthesis inhibitor F), IL-12 (NK cell stimulatory factor), MIF, IL-16, IL-17 (mCTLA-8), and/or IL-18 (IGIF, interferon-γ inducing factor)].

Additionally, certain cell surface molecules are highly expressed in tumor cells, including hormone receptors such as human chorionic gonadotropin receptor and gonadotropin releasing hormone receptor (Nechushtan et al., 1997). Therefore, the corresponding hormones may be used as the cell-specific targeting moieties in cancer therapy. Examples of hormones that may be employed as cell-specific targeting moieties include proteins, peptides, and modified amino acids, or steroids, for example. Specific hormones include human chorionic gonadotropin, gonadotropin releasing hormone, androgens, such as testosterone, for example, or estrogens, such as estradiol, for example. Additional specific hormones include thyroid-stimulating hormone, follicle-stimulating hormone, luteinizing hormone, prolactin, growth hormone, adrenocorticotropic hormone, antidiuretic hormone, oxytocin, thyrotropin-releasing hormone, growth hormone releasing hormone, corticotropin-releasing hormone, somatostatin, dopamine, melatonin, thyroxine, calcitonin, parathyroid hormone, glucocorticoids (such as cortisol, for example), mineralocorticoids (such as aldosterone, for example), adrenaline, noradrenaline, progesterone, insulin, glucagon, amylin, erythropoitin, calcitriol, calciferol, atrial-natriuretic peptide, gastrin, secretin, cholecystokinin, neuropeptide Y, ghrelin, PYY.sub.3-36, Insulin-like growth factor-1, leptin, thrombopoietin, or angiotensinogen, for example.

In addition, interferons may be employed as cell targeting moieties. Interferons (IFNs) belong to the large class of glycoproteins referred to as cytokines and are proteins generated by the immune system cells in response to challenges by foreign agents including viruses, bacteria, parasites and tumor cells, for example. Three major classes exist in humans: type I, type II, and type III. Type I IFNs comprise at least thirteen different alpha isoforms IFNA (1, 2, 4, 5, 6, 7, 8, 10, 13, 14, 16, 17, 21); a beta (IFNB1); an omega (IFNW1); an epsilon (IFNE1); and kappa (IFNK) isoforms. Type II IFNs comprise IFN gamma (IFNG). A third class comprises IFN-lambda having at least 3 different isoforms (IL29, IL28A, and IL28B).

Furthermore, vitamins may be utilized as cell-targeting moieties, including folate, vitamin D₃, vitamin K₁, vitamin E, and/or vitamin A, for example. Thus, in some embodiments of the invention, no antibodies are utilized in the chimeric polypeptides.

### C. ANTI-CELL PROLIFERATION MOIETIES

### 1. Ribosome-Inhibitory Toxins

In certain particular embodiments of the methods set forth herein, the anti-cell proliferation moiety is a ribosome-inhibitory toxin (RIT). RITs are potent inhibitors of protein synthesis in eukaryotes. The enzymatic domain of these proteins acts as a cytotoxic n-glycosidase that is able to inactivate catalytically ribosomes once they gain entry to the intracellular compartment. This is accomplished by cleaving the n-glycosidic bond of the adenine at position 4324 in the 28srRNA, which irreversibly inactivates the ribosome apparently by disrupting the binding site for elongation factors. RITs, which have been isolated from bacteria, are prevalent in higher plants. In plants, there are two types: Type I toxins possess a single polypeptide chain that has ribosome inhibiting activity, and Type II toxins have an A chain, comparable to the Type I protein, that is linked by a disulfide bond to a B chain possessing cell-binding properties. Examples of Type I RITs are gelonin, dodecandrin, tricosanthin, tricokirin, bryodin, mirabilis antiviral protein, barley ribosome-inactivating protein (BRIP), pokeweed antiviral proteins (PAPs), saporins, luffins, and momordins. Type II toxins include ricin and abrin. Toxins may be conjugated or expressed as a fusion protein with any of the polypeptides discussed herein.

### 2. Other Toxins

Any toxin known to those of ordinary skill in the art is suitable as an anti-cell proliferation moiety. Exemplary toxins include ricin A-chain (Burbage, 1997), diphtheria toxin A (Massuda et al., 1997; Lidor, 1997), pertussis toxin A subunit, E. coli enterotoxin toxin A subunit, cholera toxin A subunit and Pseudomonas toxin c-terminal are suitable. It has demonstrated that transfection of a plasmid containing the fusion protein regulatable diphtheria toxin A chain gene was cytotoxic for cancer cells. Other exemplary toxins envisioned as useful for the present invention include Abrin, A/B heat labile toxins, Botulinum toxin, Helix pomatia, Jacalin or Jackfruit, Peanut agglutinin, Sambucus nigra, Tetanus, Ulex, and Viscumin.

### 3. Recombinant Cytotoxic Agents

In some embodiments of the invention, recombinant cytotoxic agents are employed as anti-cell proliferation moieties. These may be referred to as “designer toxins.” These recombinant cytotoxic agents may be provided that is altered with respect to the native sequence, such as by having amino acids replaced or removed as compared to the native protein sequence. The recombinant cytotoxic agent may comprise the whole sequence or a partial sequence, and the partial sequence may be associated with a heterologous sequence.

For example, as indicated in U.S. patent application Ser. No. 10/074,596, which is incorporated by reference herein in its entirety, a recombinant gelonin toxin is provided that is altered with respect to the native gelonin sequence. The recombinant gelonin toxin or the present invention does not have all of the amino acids of the native gelonin, but in some embodiments, comprises a core toxin region defined as amino acid residues 110-210 of a particular sequence therein.

As an exemplary embodiment only, a recombinant gelonin toxin of the invention may include a gelonin toxin that is truncated with respect to the native sequence, such that the toxin is lacking at least 5, 10, 20, 30, 40, 50, or more amino acids. In some embodiments of the invention, the toxin contains the core toxin region, but is missing amino acids anywhere outside the core toxin region. In addition to deletions, the recombinant gelonin toxin of the invention may have an amino acid in place of a removed amino acid. For example, the glycine residue at position 7 in the gelonin protein sequence may be replaced with a non-glycine amino acid residue or a modified amino acid. If the glycine residue at position 7 is merely removed, the alanine at position 8 in SEQ ID NO: 1 becomes position 7, but is not considered a replacement because the positions of the amino acids are simply shifted by 1 position. It is contemplated that at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more amino acids may be replaced in the exemplary gelonin embodiment of the cytotoxic agent.

### 4. Apoptosis-Inducing Moieties

The present invention contemplates inclusion of any apoptosis-inducing moiety known to those of ordinary skill in the art. Exemplary pro-apoptotic amino acid sequences include CD95, caspase-3, Bax, Bag-1, CRADD, TSSC3, bax, hid, Bak, MKP-7, PERP, bad, bcl-2, MST1, bbc3, Sax, BIK, BID, and mda7. One of ordinary skill in the art would be familiar with pro-apoptotic amino acid sequences, and other such sequences not specifically set forth herein that can be applied in the methods and compositions of the present invention.

### 5. Anti-Angiogenic Amino Acid Sequences

Other examples of anti-cell proliferation moieties include anti-angiogenic amino acid sequences. An anti-angiogenic amino acid sequence is defined herein to refer to any amino acid sequence that inhibits the development of or promotes regression of angiogenesis. To the extent that these sequences can be used to target vascular endothelial cells, some of these sequences can also function as vascular endothelial targeting amino acid sequences in the context of the present invention.

Examples of anti-angiogenic amino acid sequences are set forth in U.S. Patent App. Pub. No. 20030082159 and U.S. Patent App. Pub. No. 20020114783, each of which is herein incorporated by reference in their entirety for this section of the specification and all other sections of the specification.

Any anti-angiogenic amino acid sequence known to those of ordinary skill in the art is contemplated for inclusion in the present invention. Examples of such sequences include, but are not limited to the following:

a. Tissue Inhibitors of Metalloproteinases

The tissue inhibitors of metalloproteinases (TIMPs) represent a family of ubiquitous proteins that are natural inhibitors of the matrix metalloproteinases (MMPs). Matrix metalloproteinases are a group of zinc-binding endopeptidases involved in connective tissue matrix remodeling and degradation of the extracellular matrix (ECM), an essential step in tumor invasion, angiogenesis, and metastasis. The matrix metalloproteinases each have different substrate specificities within the extracellular matrix and are important in its degradation. The analysis of matrix metalloproteinases in human mammary pathology showed that several matrix metalloproteinases were involved in degradation of the extracellular matrix: collagenase (MMP1) degrades fibrillar interstitial collagens; gelatinase (MMP2) mainly degrades type IV collagen; and stromelysin (MMP3) has a wider range of action.

There are four members of the TIMP family. TIMP-1 and TIMP-2 are capable of inhibiting tumor growth, invasion, and metastasis that has been related to matrix metalloproteinase inhibitory activity. Furthermore, both TIMP-1 and TIMP-2 are involved in the inhibition of angiogenesis. Unlike other members of the TIMP family, TIMP-3 is found only in the ECM and may function as a marker for terminal differentiation. Finally, TIMP-4 is thought to function in a tissue-specific fashion in extracellular matrix hemostasis (Gomez et al., 1997).

Tissue inhibitor of metalloproteinase-1 (TIMP-1) is a 23 kD protein that is also known as metalloproteinase inhibitor 1, fibroblast collagenase inhibitor, collagenase inhibitor and erythroid potentiating activity (EPA). The gene encoding TIMP-1 has been described by Docherty et al. (1985). TIMP-1 complexes with metalloproteinases (such as collagenases) and causes irreversible inactivation. The effects of TIMP-1 have been investigated in transgenic mouse models: one that overexpressed TIMP-1 in the liver, and another that expressed the viral oncogene Simian Virus 40/T antigen (TAg) leading to heritable development of hepatocellular carcinomas. In double transgenic experiments in which the TIMP-1 lines were crossed with the TAg transgenic line, overexpression of hepatic TIMP-1 was reported to block the development of TAg-induced hepatocellular carcinomas by inhibiting growth and angiogenesis (Martin et al., 1996).

Tissue inhibitor of metalloproteinase-2 (TIMP-2) is a 24 kD protein that is also known as metalloproteinase inhibitor 2. The gene encoding TIMP-2 has been described by Stetler-Stevenson et al. (1990). Metalloproteinase (MMP2) which plays a critical role in tumor invasion is complexed and inhibited by TIMP-2. Thus, TIMP-2 could be useful to inhibit cancer metastasis (Musso et al., 1997). When B16F10 murine melanoma cells, a highly invasive and metastatic cell line, were transfected with a plasmid coding for human TIMP-2 and injected subcutaneously in mice, TIMP-2 over-expression limited tumor growth and neoangiogenesis in vivo (Valente et al., 1998).

Tissue inhibitor of metalloproteinase-3 (TIMP-3) is also known as metalloproteinase inhibitor 3. When breast carcinoma and malignant melanoma cell lines were transfected with TIMP-3 plasmids and injected subcutaneously into nude mice, suppression of tumor growth was observed (Anand-Apte et al., 1996). However, TIMP-3 over-expression had no effect on the growth of the two tumor cell lines in vitro. Thus, it was suggested that the TIMP-3 released to the adjacent extracellular matrix by tumor cells inhibited tumor growth by suppressing the release of growth factors sequestered in extracellular matrix, or by inhibiting angiogenesis (Anand-Apte et al., 1996).

Tissue inhibitor of metalloproteinase-4 (TIMP-4) is also known as metalloproteinase inhibitor 4. The TIMP-4 gene and tissue localization have been described by Greene et al. (1996). Biochemical studies have shown that TIMP-4 binds human gelatinase A similar to that of TIMP-2 (Bigg et al., 1997). The effect of TIMP-4 modulation on the growth of human breast cancers in vivo was investigated by Wang et al. (1997). Overexpression of TIMP-4 was found to inhibit cell invasiveness in vitro, and tumor growth was significantly reduced following injection of nude mice with TIMP-4 tumor cell transfectants in vivo (Wang et al., 1997).

b. Endostatin, Angiostatin, PEX, Kringle-5

Boehm et al. (1997) showed that treatment of mice with Lewis lung carcinomas with the combination of endostatin and angiostatin proteins induced complete regression of the tumors, and that mice remained healthy for the rest of their life. This effect was obtained only after one cycle (25 days) of endostatin and angiostatin treatment, whereas endostatin alone required 6 cycles to induce tumor dormancy.

Bergers et al. (1999) demonstrated a superior antitumoral effect of the combination of endostatin and angiostatin proteins in a mouse model for pancreatic islet carcinoma. Endostatin and angiostatin combination resulted in a significant regression of the tumors, whereas endostatin or angiostatin alone had no effect.

Endostatin, an angiogenesis inhibitor produced by hemangioendothelioma, was first identified by O'Reilly et al. (1997). Endostatin is a 20 kD C-terminal fragment of collagen XVIII that specifically inhibits endothelial proliferation, and potently inhibits angiogenesis and tumor growth. In fact, primary tumors have been shown to regress to dormant microscopic lesions following the administration of recombinant endostatin (O'Reilly et al., 1997). Endostatin is reported to inhibit angiogenesis by binding to the heparin sulfate proteoglycans involved in growth factor signaling (Zetter, 1998).

Recently, a C-terminal fragment of collagen XV (Endostatin XV) has been shown to inhibit angiogenesis like Endostatin XVIII, but with several functional differences (Sasaki et al., 2000).

Angiostatin, an internal fragment of plasminogen comprising the first four kringle structures, is one of the most potent endogenous angiogenesis inhibitors described to date. It has been shown that systemic administration of angiostatin efficiently suppresses malignant glioma growth in vivo (Kirsch et al., 1998). Angiostatin has also been combined with conventional radiotherapy resulting in increased tumor eradication without increasing toxic effects in vivo (Mauceri et al., 1998). Other studies have demonstrated that retroviral and adenoviral mediated gene transfer of angiostatin cDNA resulted in inhibition of endothelial cell growth in vitro and angiogenesis in vivo. The inhibition of tumor-induced angiogenesis produced an increase in tumor cell death (Tanaka et al., 1998). Gene transfer of a cDNA coding for mouse angiostatin into murine T241 fibrosarcoma cells has been shown to suppress primary and metastatic tumor growth in vivo (Cao et al., 1998).

PEX is the C-terminal hemopexin domain of MMP-2 that inhibits the binding of MMP-2 to integrin αvβ3 and blocks cell surface collagenolytic activity required for angiogenesis and tumor growth. It was cloned and described by Brooks et al. (1994).

The kringle-5 domain of human plasminogen, which shares high sequence homology with the four kringles of angiostatin, has been shown to be a specific inhibitor for endothelial cell proliferation. Kringle-5 appears to be more potent than angiostatin on inhibition of basic fibroblast growth factor-stimulated capillary endothelial cell proliferation (Cao et al., 1997). In addition to its antiproliferative properties, kringle-5 also displays an anti-migratory activity similar to that of angiostatin that selectively affects endothelial cells (Ji et al., 1998).

c. Chemokines

Chemokines are low-molecular weight pro-inflammatory cytokines capable of eliciting leukocyte chemotaxis. Depending on the chemokine considered, the chemoattraction is specific for certain leukocyte cell types. Moreover, in addition to their chemotactic activity, some chemokines possess an anti-angiogenic activity, i.e. they inhibit the formation of blood vessels feeding the tumor. For this reason, these chemokines are useful in cancer treatment.

d. Monokine-Induced by Interferon-Gamma (MIG)

MIG, the monokine-induced by interferon-gamma, is a CXC chemokine related to IP-10 and produced by monocytes. MIG is a chemoattractant for activated T cells, and also possesses strong angiostatic properties. Intratumoral injections of MIG induced tumor necrosis (Sgadari et al., 1997).

e. Interferon-Alpha Inducible Protein 10 (IP-10)

IP-10, the interferon-alpha inducible protein 10, is a member of the CXC chemokine family. IP-10 is produced mainly by monocytes, but also by T cells, fibroblasts and endothelial cells. IP-10 exerts a chemotactic activity on lymphoid cells such as T cells, monocytes and NK cells. IP-10 is also a potent inhibitor of angiogenesis. It inhibits neovascularization by suppressing endothelial cell differentiation. Because of its chemotactic activity toward immune cells, IP-10 was considered as a good candidate to enhance antitumour immune responses. Gene transfer of IP-10 into tumor cells reduced their tumorigenicity and elicited a long-term protective immune response (Luster and Leder, 1993). The angiostatic activity of IP-10 was also shown to mediate tumor regression. Tumor cells expressing IP-10 became necrotic in vivo (Sgadari et al., 1996). IP-10 was also shown to mediate the angiostatic effects of IL-12 that lead to tumor regression (Tannenbaum et al., 1998).

f. VEGF Receptors

FLT-1 (fms-like tyrosine kinase 1 receptor) is a membrane-bound receptor of VEGF (VEGF Receptor 1). It has been shown that a soluble fragment of FLT-1 (sFLT-1) has angiostatic properties by way of its antagonist activity against VEGF. Soluble FLT-1 acts by binding to VEGF but also because it binds and blocks the external domain of the membrane-bound FLT-1. One example of sFLT-1 is a human sFLT-1 spanning the 7 immunoglobulin-like domains of the external part of FLT-1.

g. sFLK-1/KDR

FLK-1 or KDR (kinase insert domain receptor) is a membrane-bound receptor of VEGF (VEGF Receptor 2). It has been shown that a soluble fragment of KDR (sKDR) has angiostatic properties by way of its antagonist activity against VEGF. The sKDR also binds and blocks the external domain of the membrane-bound KDR. One example of sKDR is a human sKDR spanning the 7 immunoglobulin-like domains of the external part of KDR.

### D. MUTEINS

In particular embodiments, an altered molecule, such as an altered TNF molecule, including an altered TNF-.alpha. molecule, is employed in the compounds of the present invention. Other molecules may be employed as muteins, and TNF is described herein merely as an exemplary embodiment. The altered TNF molecule may be further defined as a mutant of TNF, which may be even further defined as a TNF mutein. A TNF mutein comprises a TNF molecule having one or more mutations, wherein the TNF molecule retains TNF function, which in specific embodiments refers to being cytotoxic to a cancer cell. In specific embodiments, the TNF mutein comprises substantially the same or greater activity than wild-type TNF, such as concerning anti-cancer activity and low toxicity. The alteration may affect the binding affinity of TNF to p75-TNF-receptor and/or to p55-TNF-receptor.

In embodiments of the invention, the mutein is altered by substitution of one or more amino acids and in specific embodiments is by naturally occurring amino acids.

The one or more mutations may be in the N-terminus and/or the C-terminus, for example. The mutation may be a point mutation, a frame shift mutation, a deletion, an inversion, or a splicing mutant, for example. The mutation may be in a particular region of TNF, such as a functional domain of TNF. In specific embodiments, the mutation is in the trimerization domain. An exemplary TNF molecule for alteration to a TNF mutein is provided in SEQ ID NO:10 (GenBank Accession No. AAA61200). TNF-muteins may be designed based on the 3-D structure of the protein and molecular modelling approaches.

Specific examples of TNF muteins include those identified, for example, in U.S. Pat. No. 5,773,582; U.S. Pat. No. 5,422,104; U.S. Pat. No. 5,247,070; U.S. Pat. No. 5,606,023; U.S. Pat. No. 5,652,353; U.S. Pat. No. 4,677,064; U.S. Pat. No. 5,519,119; and U.S. Pat. No. 5,652,353, all of which are incorporated by reference herein in their entirety.

### E. CHELATOR MOIETIES

The pharmaceutical compounds set forth herein include one or more chelator moieties. The term “moiety” as used herein refers to a part of the compound of the present invention. The “chelating moiety” can be any chelating agent known to those of ordinary skill in the art. A “chelator” is any substance that binds particular ions.

### 1. Examples of Chelator Moieties

Other examples of chelator moieties include, but are not limited to, DTPA (diethylenetriamine pentaacetic acid); dimercaptosuccinic acid (DMSA); ethylenediaminetetraacetic acid (EDTA); 1,2-cyclohexanediamine-N,N,N′,N′-tetraacetic acid (Cy-EDTA); ethylenediaminetetramethylenephosphonic acid (EDTMP); N-[2-[bis(carboxymethyl)amino]cyclohexyl]-N′-(carboxymethyl)-N,N′-ethylenediglycine (CyDTPA); N,N-bis[2-[bis(carboxymethyl)amino]cyclohexylglycine (Cy.sub.2DTPA); 2,5,8-tris(carboxymethyl)-12-phenyl-11-oxa-2,5,8-triazadodecane-1,9-dicarboxylic acid (BOPTA); diethylenetriaminepentaacetic acid, monoamide (DTPA-MA); diethylenetriaminepentaacetic acid, biamide (DTPA-BA); diethylenetriamine-N,N,N′,N″,N″-pentamethylenephosphonic acid (DTPMP); tetraazacyclododecane-N,N′,N″,N″-tetraacetic acid (DOTA); tetraazacyclotridecane-N,N′,N″,N″-tetraacetic acid (TRITA); tetraazacyclotetradecane-N,N′,N″,N″-tetraacetic acid (TETA); tetraazacyclododecane-α,α′,α″,α′″-tetramethyl-N,N′,N″,N′″-tetraacetic acid (DOTMA); tetraazacyclododecane-N,N′,N″,N′″-tetra-acetic acid, monoamide (DOTA-MA); 1042-hydroxypropyl)-1,4,7,10-tetraazacy-clododecane-1,4,7-triacetic acid (HP-DO3A); 1-((p-nitrophenyl)carboxymethyl-1)-4,7,10-tris(carboxymethyl)-1,4,7,10-tetraazacyclododecane (pNB-DOTA); tetraazacyclodecane-N,N′,N″,N″-tekamethylenephosphonic acid (DOTP); tetraazacyclododecane-N,N′,N″,N′″-tetramethylenetetramethylphosphinic acid (DOTMP); tetraazacyclododecane-N,N′,N″,N″-tetramethylenetetraethylphosphinic acid (DOTEP); tetraazacyclododecane-N,N′,N″,N′″-tetramethylenete-traphenylphosphinic acid (DOTPP); tetraazacyclododecane-N,N′,N″,N′″-tetramethylenetetrabenzylphosphinic acid (DOTBzP); tetraazacyclodecane-N,N′,N″,N′″-tetramethylenephosphonic acid-P,P′,P″,P′″-tetraethyl ester (DOTPME); hydroxyethylidenediphosphonate (HEDP); diethylenetriaminetetramethyleneph-osphonic acid (DTTP); N.sub.3S triamidethiols (MAG3); N₂S₂ diamidedithiols (DADS); N₂S₂ monoamidemonoaminedithiols (MAMA); N₂S₂ diaminedithiols (DADT); N₂S₂ diaminedithiols acid (ethyleledicysteine, EC), N₂S₄ diaminetetrathiols; N₂P₂ dithiol-bisphosphines; 6-hydrazinonicotinic acids; propylene amine oximes; tetraamines; cyclals and cyclams. Another example is ethylenedicysteine. One of ordinary skill in the art would be familiar with the numerous agents that can be applied as chelator moieties in the context of the present invention.

The chelator moieties of the present invention may comprise a combination of any number of atoms selected from the group consisting of nitrogen atoms, sulfur atoms, oxygen atoms, and phosphorus atoms. In certain particular embodiments, the chelator moiety includes a combination of three to five such atoms. In some embodiments, the chelator is capable of chelating to any number of valent metal ions through coordination to other atoms, such as nitrogen atoms, sulfur atoms, oxygen atoms, and/or phosphorus atoms. In certain embodiments, the chelator is capable of chelating three to five valent metal ions. Any valent metal ion is contemplated for chelation to the chelators of the present invention. Examples of these valent metal ions include, but are not limited to, Tc-99m, Cu-60, Cu-61, Cu-62, Cu-64, In-111, Tl-201, Ga-67, and Ga-68.

In certain embodiments of the present invention, the compounds of the present invention include chelator moieties wherein the chelator is capable of chelating to a beta-emitter. Examples of beta emitters include Re-188, Re-186, Sr-89, Ho-166, Y-90, Sn-153, As-72. One of ordinary skill in the art would be familiar with additional such beta emitters that can be applied to the delivery of radiotherapy, which is discussed in greater detail in other parts of this specification.

### 2. Conjugation of Chelator Moiety to a Cell-Specific Targeting Moiety

Any method known to those of ordinary skill in the art can be used to conjugate a chelator moiety to a cell-specific targeting moiety. For example, an aqueous solution of the cell-specific targeting moiety conjugated to the anti-cell proliferation moiety is prepared. To this solution, a coupling agent and a chelator is added. The reaction mixture can be stirred at room temperature for any period of time. The conjugate can then be isolated from solution using any method known to those of skill in the art. For example, in some embodiments, the product is isolated from solution by dialysis. The resulting compound of the present invention can either be used immediately, or stored for later use. One or more purification steps can optionally be employed.

In other embodiments, the chelator and the anti-cell proliferation moiety are conjugated to the carbohydrate in the same reaction mixture using a linker. Any coupling agent known to those of ordinary skill in the art can be used. Linkers are discussed in greater detail below.

### F. LINKERS

The pharmaceutical compounds of the present invention may be produced in any suitable manner in the art, although in particular embodiments the compound is generated using a linker to attach the cell-specific targeting moiety to the anti-cell proliferation moiety. As used herein, a “linker” is a chemical or peptide or polypeptide that links an endothelial targeting amino acid sequence with a cytotoxic amino acid sequence.

The two coding sequences can be fused directly without any linker or by using a flexible polylinker, such as one composed of the pentamer Gly-Gly-Gly-Gly-Ser (SEQ ID NO:9) repeated 1 to 3 times. Such linker has been used in constructing single chain antibodies (scFv) by being inserted between V_{H} and V_{L} (Bird et al., 1988; Huston et al., 1988). The linker is designed to enable the correct interaction between two beta-sheets forming the variable region of the single chain antibody. Other linkers which may be used include Glu-Gly-Lys-Ser-Ser-Gly-Ser-Gly-Ser-Glu-Ser-Lys-Val-Asp (SEQ ID NO:1) (Chaudhary et al., 1990) and Lys-Glu-Ser-Gly-Ser-Val-Ser-Ser-Glu-Gln-Leu-Ala-Gln-Phe-Arg-Ser-Leu-Asp (SEQ ID NO:11) (Bird et al., 1988).

Multiple peptides or polypeptides may also be joined via a biologically-releasable bond, such as a selectively-cleavable linker or amino acid sequence. For example, peptide linkers that include a cleavage site for an enzyme preferentially located or active within a tumor environment are contemplated. Exemplary forms of such peptide linkers are those that are cleaved by urokinase, plasmin, thrombin, Factor IXa, Factor Xa, or a metallaproteinase, such as collagenase, gelatinase, or stromelysin. Alternatively, polypeptides may be joined to an adjuvant. It can be considered as a general guideline that any linker known to those of ordinary skill in the art is contemplated for use as a linker in the present invention.

It is contemplated that cross-linkers may be implemented with the polypeptide molecules of the present invention. Cross-linking reagents are used to form molecular bridges that tie together functional groups of two different molecules, e.g., a stablizing and coagulating agent. To link two different polypeptides in a step-wise manner, hetero-bifunctional cross-linkers can be used that eliminate unwanted homopolymer formation. Bifunctional cross-linking reagents have been extensively used for a variety of purposes including preparation of affinity matrices, modification and stabilization of diverse structures, identification of binding sites, and structural studies. In the context of the invention, such cross-linker may be used to stabilize the polypeptide or to render it more useful as a therapeutic, for example, by improving the polypeptide's targeting capability or overall efficacy. Cross-linkers may also be cleavable, such as disulfides, acid-sensitive linkers, and others. Homobifunctional reagents that carry two identical functional groups proved to be highly efficient in inducing cross-linking between identical and different macromolecules or subunits of a macromolecule, and linking of polypeptides to specific binding sites on binding partners. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino, sulfhydryl, guanidino, indole, carboxyl specific groups. Of these, reagents directed to free amino groups have become especially popular because of their commercial availability, ease of synthesis and the mild reaction conditions under which they can be applied. A majority of heterobifunctional cross-linking reagents contains a primary amine-reactive group and a thiol-reactive group.

The linker may be a peptide linker. Peptide linkers that include a cleavage site for an enzyme preferentially located or active within a tumor environment are contemplated. Exemplary forms of such peptide linkers are those that are cleaved by urokinase, plasmin, thrombin, Factor IXa, Factor Xa, or a metallaproteinase, such as collagenase, gelatinase, or stromelysin. Alternatively, peptides or polypeptides may be joined to an adjuvant.

Amino acids such as selectively-cleavable linkers, synthetic linkers, or other amino acid sequences may be used to separate proteinaceous moieties. Additionally, while numerous types of disulfide-bond containing linkers are known that can successfully be employed to conjugate the toxin moiety with the targeting agent, certain linkers will generally be preferred over other linkers, based on differing pharmacologic characteristics and capabilities. For example, linkers that contain a disulfide bond that is sterically “hindered” are to be preferred, due to their greater stability in vivo, thus preventing release of the toxin moiety prior to binding at the site of action.

Additionally, any other linking/coupling agents and/or mechanisms known to those of skill in the art can be used to combine the components of the present invention, such as, for example, antibody-antigen interaction, avidin biotin linkages, amide linkages, ester linkages, thioester linkages, ether linkages, thioether linkages, phosphoester linkages, phosphoramide linkages, anhydride linkages, disulfide linkages, ionic and hydrophobic interactions, bispecific antibodies and antibody fragments, or combinations thereof.

It is contemplated that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido)ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art. Another embodiment involves the use of flexible linkers.

U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Preferred uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

In another example, heterobifunctional cross-linking reagents and methods of using the cross-linking reagents are described (U.S. Pat. No. 5,889,155, specifically incorporated herein by reference in its entirety). The cross-linking reagents combine a nucleophilic hydrazide residue with an electrophilic maleimide residue, allowing coupling in one example, of aldehydes to free thiols. The cross-linking reagent can be modified to cross-link various functional groups and is thus useful for cross-linking polypeptides and sugars. In instances where a particular polypeptide, such as gelonin, does not contain a residue amenable for a given cross-linking reagent in its native sequence, conservative genetic or synthetic amino acid changes in the primary sequence can be utilized. Table 2 details certain exemplary hetero-bifunctional cross-linkers considered useful in the present invention.

TABLE 2
HETERO-BIFUNCTIONAL CROSS-LINKERS

| | | | Spacer Arm |
|---|---|---|---|
| | | | Length\after |
| Linker | Reactive Toward | Advantages and Applications | cross-linking |
| SMPT | Primary amines | Greater stability | 11.2 A |
| | Sulfhydryls | | |
| SPDP | Primary amines | Thiolation | 6.8 A |
| | Sulfhydryls | Cleavable cross-linking | |
| LC-SPDP | Primary amines | Extended spacer arm | 15.6 A |
| | Sulfhydryls | | |
| Sulfo-LC- | Primary amines | Extended spacer arm | 15.6 A |
| SPDP | Sulfhydryls | Water-soluble | |
| SMCC | Primary amines | Stable maleimide reactive group | 11.6 A |
| | Sulfhydryls | Enzyme-antibody conjugation | |
| | | Hapten-carrier protein conjugation | |
| Sulfo- | Primary amines | Stable maleimide reactive group | 11.6 A |
| SMCC | Sulfhydryls | Water-soluble | |
| | | Enzyme-antibody conjugation | |
| MBS | Primary amines | Enzyme-antibody conjugation | 9.9 A |
| | Sulfhydryls | Hapten-carrier protein conjugation | |
| Sulfo- | Primary amines | Water-soluble | 9.9 A |
| MBS | Sulfhydryls | | |
| SIAB | Primary amines | Enzyme-antibody conjugation | 10.6 A |
| | Sulfhydryls | | |
| Sulfo- | Primary amines | Water-soluble | 10.6 A |
| SIAB | Sulfhydryls | | |
| SMPB | Primary amines | Extended spacer arm | 14.5 A |
| | Sulfhydryls | Enzyme-antibody conjugation | |
| Sulfo- | Primary amines | Extended spacer arm | 14.5 A |
| SMPB | Sulfhydryls | Water-soluble | |
| EDC/Sulfo- | Primary amines | Hapten-Carrier conjugation | 0 |
| NHS | Carboxyl groups | | |
| ABH | Carbohydrates | Reacts with sugar groups | 11.9 A |
| | Nonselective | | |

### G. VALENT METAL IONS AND LABELING OF CHELATORS

In certain embodiments, a valent metal ion is chelated to the chelator moiety of the pharmaceutical compounds of the present invention. A variety of valent metal ions, or radionuclides, are known to be useful for radioimaging. Examples include ⁶⁷Ga, ⁶⁸Ga, ^{99m}Tc, ¹¹¹In, ¹²³I, ¹²⁵I, ¹³¹I, 169Yb, ⁶⁰Cu, ⁶¹Cu, ⁶²Cu, ⁶⁴Cu, ²⁰¹Tl, ⁷²A, and ¹⁵⁷Gd.

In some embodiments, the valent metal ion is a gamma emitter. A “gamma emitter” is herein defined as an agent that emits gamma energy of any range. One of ordinary skill in the art would be familiar with the various valent metal ions that are gamma emitters. In some embodiments, the valent metal ion is a beta emitter.

Any method known to those of ordinary skill in the art can be used to chelate a valent metal ion to a chelator moiety of the present pharmaceutical compounds. In some embodiments of the present invention, for example, the conjugate is dissolved in water, and then tin(II) chloride solution added. The radiolabel can then be added. Other metals (gallium chloride, gadolinium chloride, copper chloride, cobolt chloride, platinum) may not require tin(II) chloride solution. Any method known to those of ordinary skill in the art can be used to measure radiochemical purity. For example, it may be measured using thin layer chromatography (TLC) eluted with methanol:ammonium acetate (1:4).

Any method known to those of ordinary skill in the art can be used to isolate the radiolabeled conjugate from solution. For example, in some embodiments, the reaction mixture can be evaporated to dryness, and then later reconstituted in water for use.

### H. IMAGING

Certain embodiments of the present invention pertain to methods of imaging a subject following administration of a pharmaceutically effective amount of a compound as set forth herein. Any imaging modality known to those of ordinary skill in the art is contemplated by the present invention. Examples of imaging modalities are set forth as follows. Any imaging modality known to those of ordinary skill in the art is contemplated by the present invention. Examples include PET, CT, SPECT, MRI, and optical imaging. Other examples of imaging modalities include digital subtraction angiography and x-ray angiography.

### 1. Examples of Imaging Modalities

a. Computerized Tomography (CT)

Computerized tomography (CT) is contemplated as an imaging modality in the context of the present invention. By taking a series of X-rays, sometimes more than a thousand, from various angles and then combining them with a computer, CT made it possible to build up a three-dimensional image of any part of the body. A computer is programmed to display two-dimensional slices from any angle and at any depth.

In CT, intravenous injection of a radiopaque contrast agent can assist in the identification and delineation of soft tissue masses when initial CT scans are not diagnostic. Similarly, contrast agents aid in assessing the vascularity of a soft tissue or bone lesion. For example, the use of contrast agents may aid the delineation of the relationship of a tumor and adjacent vascular structures.

CT contrast agents include, for example, iodinated contrast media. Examples of these agents include iothalamate, iohexyl, diatrizoate, iopamidol, ethiodol, and iopanoate. Gadolinium agents have also been reported to be of use as a CT contrast agent (see, e.g., Henson et al., 2004). For example, gadopentate agents has been used as a CT contrast agent (discussed in Strunk and Schild, 2004).

b. Magnetic Resonance Imaging (MRI)

Magnetic resonance imaging (MRI) is an imaging modality that is newer than CT that uses a high-strength magnet and radio-frequency signals to produce images. The most abundant molecular species in biological tissues is water. It is the quantum mechanical “spin” of the water proton nuclei that ultimately gives rise to the signal in imaging experiments. In MRI, the sample to be imaged is placed in a strong static magnetic field (1-12 Tesla) and the spins are excited with a pulse of radio frequency (RF) radiation to produce a net magnetization in the sample. Various magnetic field gradients and other RF pulses then act on the spins to code spatial information into the recorded signals. By collecting and analyzing these signals, it is possible to compute a three-dimensional image which, like a CT image, is normally displayed in two-dimensional slices.

Contrast agents used in MR imaging differ from those used in other imaging techniques. Their purpose is to aid in distinguishing between tissue components with identical signal characteristics and to shorten the relaxation times (which will produce a stronger signal on T1-weighted spin-echo MR images and a less intense signal on T2-weighted images). Examples of MRI contrast agents include gadolinium chelates, manganese chelates, chromium chelates, and iron particles.

Both CT and MRI provide anatomical information that aid in distinguishing tissue boundaries and vascular structure. Compared to CT, the disadvantages of MRI include lower patient tolerance, contraindications in pacemakers and certain other implanted metallic devices, and artifacts related to multiple causes, not the least of which is motion (Alberico et al., 2004). CT, on the other hand, is fast, well tolerated, and readily available but has lower contrast resolution than MRI and requires iodinated contrast and ionizing radiation (Alberico et al., 2004). A disadvantage of both CT and MRI is that neither imaging modality provides functional information at the cellular level. For example, neither modality provides information regarding cellular viability.

c. PET and SPECT

Imaging modalities that provide information pertaining to information at the cellular level, such as cellular viability, include positron emission tomography (PET) and single-photon emission computed tomography (SPECT). In PET, a patient ingests or is injected with a slightly radioactive substance that emits positrons, which can be monitored as the substance moves through the body. In one common application, for instance, patients are given glucose with positron emitters attached, and their brains are monitored as they perform various tasks. Since the brain uses glucose as it works, a PET image shows where brain activity is high.

Closely related to PET is single-photon emission computed tomography, or SPECT. The major difference between the two is that instead of a positron-emitting substance, SPECT uses a radioactive tracer that emits high-energy photons. SPECT is valuable for diagnosing coronary artery disease, and already some 2.5 million SPECT heart studies are done in the United States each year.

PET radiopharmaceuticals for imaging are commonly labeled with positron-emitters such as ¹¹C, ¹³N, ¹⁵O, ¹⁸F, ⁸²Rb, ⁶²Cu, and ⁶⁸Ga. SPECT radiopharmaceuticals are commonly labeled with positron emitters such as ^{99m}Tc, ²⁰¹Tl, and ⁶⁷Ga. Regarding brain imaging, PET and SPECT radiopharmaceuticals are classified according to blood-brain-barrier permeability, cerebral perfusion and metabolism receptor-binding, and antigen-antibody binding (Saha et al., 1994). The blood-brain-barrier SPECT agents, such as ^{99m}TcO4-DTPA, ²⁰¹T1, and [⁶⁷Ga]citrate are excluded by normal brain cells, but enter into tumor cells because of altered BBB. SPECT perfusion agents such as [¹²³I]IMP, [^{99m}Tc]HMPAO, [^{99m}Tc]ECD are lipophilic agents, and therefore diffuse into the normal brain. Important receptor-binding SPECT radiopharmaceuticals include [¹²³I]QNE, [¹²³I]IBZM, and [¹²³I]iomazenil. These tracers bind to specific receptors, and are of importance in the evaluation of receptor-related diseases.

### 2. Imaging Technique

The radiolabeled compounds provided by the present invention can be used for visualizing sites in a subject. In accordance with this invention, the imaging agents are administered by any method known to those of ordinary skill in the art. For example, administration may be in a single unit injectable dose. Any of the common carriers known to those with skill in the art, such as sterile saline solution or plasma, may be utilized after radiolabeling for preparing the compounds of the present invention for injection.

After intravenous administration of a compound of the present invention that is an imaging agent, imaging of the organ or tumor in vivo can take place, if desired, in hours or even longer, after the radiolabeled reagent is introduced into a patient. A sufficient amount of the administered dose will accumulate in the area to be imaged within about 10 minutes in some embodiments. As set forth above, imaging may be performed using any method known to those of ordinary skill in the art. Examples include PET, SPECT, and gamma scintigraphy. In gamma scintigraphy, the radiolabel is a gamma-radiation emitting radionuclide and the radiotracer is located using a gamma-radiation detecting camera (this process is often referred to as gamma scintigraphy). The imaged site is detectable because the radiotracer is chosen either to localize at a pathological site (termed positive contrast) or, alternatively, the radiotracer is chosen specifically not to localize at such pathological sites (termed negative contrast).

### I. HYPERPROLIFERATIVE DISEASE

Certain aspects of the present invention are generally concerned with methods of treating or imaging hyperproliferative disease in a subject using a compound of the present invention. A “hyperproliferative disease” is herein defined as any disease associated with abnormal cell growth or abnormal cell turnover For example, the hyperproliferative disease may be cancer. The term “cancer” as used herein is defined as an uncontrolled and progressive growth of cells in a tissue. A skilled artisan is aware other synonymous terms exist, such as neoplasm or malignancy or tumor. Any type of cancer is contemplated for treatment by the methods of the present invention. For example, the cancer may be breast cancer, lung cancer, ovarian cancer, brain cancer, liver cancer, cervical cancer, colon cancer, renal cancer, skin cancer, head and neck cancer, bone cancer, esophageal cancer, bladder cancer, uterine cancer, stomach cancer, pancreatic cancer, testicular cancer, lymphoma, or leukemia. In other embodiments of the present invention, the cancer is metastatic cancer. In other embodiments, the hyperproliferative disease is a disease associated with neovascularization that is noncancerous.

### J. ADMINISTRATION

### 1. Routes of Administration

In some embodiments, an effective amount of a pharmaceutical compound of the present invention is administered to a subject. Administration may be for the purpose of imaging and/or for the purpose of treating a hyperproliferative disease in a subject.

The term “effective amount” as used herein is defined as the amount of the compound of the present invention that is necessary to result in a physiological change in the cell or tissue to which it is administered. The term “therapeutically effective amount” as used herein is defined as the amount of the compound of the present invention that eliminates, decreases, delays, or minimizes adverse effects of a disease, such as cancer. The term “diagnostically effective amount” as used herein refers to that amount of the compound that when administered would be known or suspected to result in a detectable signal from a site in the subject if a hyperproliferative disease is present at the site.

A skilled artisan readily recognizes that in many cases the compounds set forth herein may not provide a cure but may only provide partial benefit, such as alleviation or improvement of at least one symptom. In some embodiments, a physiological change having some benefit is also considered therapeutically beneficial. Thus, in some embodiments, an amount of chimeric molecules that provides a physiological change is considered an “effective amount” or a “therapeutically effective amount.”

The pharmaceutical compounds of the invention may be administered to a subject per se or in the form of a pharmaceutical composition for the treatment of a hyperproliferative disease, such as cancer.

Pharmaceutical compositions comprising the proteins of the invention may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries that facilitate processing of the proteins into preparations which can be used pharmaceutically. Proper formulation is dependent upon the route of administration chosen.

Systemic formulations include those designed for administration by injection, e.g. subcutaneous, intravenous, intramuscular, intrathecal or intraperitoneal injection, as well as those designed for transdermal, transmucosal, inhalation, oral or pulmonary administration. Topical application is also contemplated by the present invention.

For injection, the proteins of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents.

Alternatively, the proteins may be in powder form for constitution with a suitable vehicle, e.g., sterile pyrogen-free water, before use.

For transmucosal administration, penetrants appropriate to the barrier to be permeated are used in the formulation. Such penetrants are generally known in the art.

For oral administration, the proteins can be readily formulated by combining the proteins with pharmaceutically acceptable carriers well known in the art. Such carriers enable the proteins of the invention to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, e.g. lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate.

If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, etc. Additionally, flavoring agents, preservatives, coloring agents and the like may be added.

For buccal administration, the molecules may take the form of tablets, lozenges, etc. formulated in conventional manner.

For administration by inhalation, the molecules for use according to the present invention are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator may be formulated containing a powder mix of the protein and a suitable powder base such as lactose or starch.

In addition to the formulations described previously, the molecules may also be formulated as a depot preparation. Such long acting formulations may be administered by implantation (for example subcutaneously or intramuscularly) or by intramuscular injection. Thus, for example, the molecules may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems may be employed. Liposomes and emulsions are well-known examples of delivery vehicles that may be used to deliver proteins of the invention. Certain organic solvents such as dimethylsulfoxide also may be employed, although usually at the cost of greater toxicity. Additionally, the molecules may be delivered using a sustained-release system, such as semipermeable matrices of solid polymers containing the therapeutic agent. Various of sustained-release materials have been established and are well known by those skilled in the art. Sustained-release capsules may, depending on their chemical nature, release the molecules for a few weeks up to over 100 days. Depending on the chemical nature and the biological stability of the chimeric molecules, additional strategies for molecule stabilization may be employed.

As the protein embodiments of the chimeric molecules of the invention may contain charged side chains or termini, they may be included in any of the above-described formulations as the free acids or bases or as pharmaceutically acceptable salts. Pharmaceutically acceptable salts are those salts that substantially retain the biologic activity of the free bases and which are prepared by reaction with inorganic acids. Pharmaceutical salts tend to be more soluble in aqueous and other protic solvents than are the corresponding free base forms.

### 2. Effective Dosages

The pharmaceutical compounds of the invention will generally be used in an amount effective to achieve the intended purpose. For use to image a site of disease in a subject, the pharmaceutical compound is administered or applied in a diagnostically effective amount to image the site. A therapeutically effective amount is an amount effective to ameliorate or prevent the symptoms, or prolong the survival of, the patient being treated. Determination of a therapeutically effective amount or diagnostically effective amount is well within the capabilities of those skilled in the art, especially in light of the detailed disclosure provided herein.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the molecules which are sufficient to maintain therapeutic effect. Usual patient dosages for administration by injection range from about 0.1 to 5 mg/kg/day, preferably from about 0.5 to 1 mg/kg/day. Therapeutically effective serum levels may be achieved by administering multiple doses each day.

In cases of local administration or selective uptake, the effective local concentration of the proteins may not be related to plasma concentration. One having skill in the art will be able to optimize therapeutically effective local dosages without undue experimentation.

The amount of molecules administered will, of course, be dependent on the subject being treated, on the subject's weight, the severity of the affliction, the manner of administration and the judgment of the prescribing physician.

### K. PHARMACEUTICAL PREPARATIONS

Pharmaceutical compositions of the present invention comprise an effective amount of one or more pharmaceutical compounds of the present invention and, in some embodiments, at least one additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one chimeric polypeptide or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The pharmaceutical compositions may comprise different types of carriers depending on whether it is to be administered in solid, liquid or aerosol form, and whether it needs to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g. aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The pharmaceutical compound may be formulated into a composition in a free base, neutral or salt form. Pharmaceutically acceptable salts, include the acid addition salts, e.g., those formed with the free amino groups of a proteinaceous composition, or that are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments, the compounds are prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

### L. COMBINATION TREATMENTS/CANCER THERAPIES

In order to increase the effectiveness of a pharmaceutical compound of the present invention, or expression construct coding therefor, it may be desirable to combine these compositions with other agents effective in the treatment of hyperproliferative disease, such as anti-cancer agents. Indeed, in particular embodiments, the compounds set forth herein are employed with one or more chemotherapeutic agents, such as to render effective the chemotherapeutic agent on a resistant cell. The compound alone or in conjunction with one or more chemotherpeutic agents may be administered to an individual with cancer in addition to another cancer therapy, such as radiation, surgery, gene therapy, and so forth.

An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with gene therapy. For example, the herpes simplex-thymidine kinase (HS-tK) gene, when delivered to brain tumors by a retroviral vector system, successfully induced susceptibility to the antiviral agent ganciclovir (Culver, et al., 1992). In the context of the present invention, it is contemplated that chimeric molecules could be used similarly in conjunction with chemotherapeutic, radiotherapeutic, gene therapy, or immunotherapeutic intervention, in addition to other pro-apoptotic or cell cycle regulating agents.

Alternatively, the therapy may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, wherein compound of the present invention is “A” and the secondary agent, such as radio- or chemotherapy, for example, is “B”: TABLE-US-00001 A/B/A B/A/B B/B/A A/A/B A/B/B B/A/A A/B/B/B B/A/B/B B/B/B/A B/B/A/B A/A/B/B A/B/A/B A/B/B/A B/B/A/A B/A/B/A B/A/A/B A/A/A/B B/A/A/A A/B/A/A A/A/B/A.

Administration of the therapeutic expression constructs of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described hyperproliferative cell therapy.

### 1. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

### 2. Radiotherapy

Other factors that cause DNA damage and have been used extensively include what are commonly known as 7-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

### 3. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with gene therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

### 4. Genes

In yet another embodiment, the secondary treatment is a gene therapy in which a therapeutic polynucleotide is administered before, after, or at the same time as a chimeric polypeptide of the present invention. Delivery of a chimeric polypeptide in conjuction with a second vector encoding one of the following gene products will have a combined anti-hyperproliferative effect on target tissues. Alternatively, a single vector encoding both genes may be used. A variety of proteins are encompassed within the invention, some of which are described below. These include inducers of cellular proliferation and regulators of programmed cell death. One of ordinary skill in the art would be familiar with such therapeutic genes.

### 5. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies. The chimeric molecule of the present invention may be employed as neoadjuvant surgical therapy, such as to reduce tumor size prior to resection, or it may be employed as postadjuvant surgical therapy, such as to sterilize a surgical bed following removal of part or all of a tumor.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### 6. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

### M. KITS OF THE INVENTION

Any one or more of the pharmaceutical compounds or compositions described herein may be comprised in a kit. In a non-limiting example, a pharmaceutical compound of the present invention, the components of the compound, and/or one or more additional agents may be comprised in a kit. The kits will thus comprise, in suitable container means, a pharmaceutical compound of the present invention, the pharmaceutical compound components and/or an additional agent of the present invention.

The kits may comprise a suitably aliquoted amount of pharmaceutical compound, components and/or additional agent compositions of the present invention, whether labeled or unlabeled, as may be used for treatment of one or more individuals with a hyperproliferative disease. The one or more components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one components in the kit, the kit also will generally contain a second, third or other additional container into which the additional components may be separately placed. However, various combinations of components may be comprised in a vial. The kits of the present invention also will typically include a means for containing the pharmaceutical compound, the compound components and/or additional agent, and any other reagent containers in close confinement for commercial sale. Such containers may include injection or blow molded plastic containers into which the desired vials are retained.

Therapeutic or diagnostic kits of the present invention are kits comprising the pharmaceutical compound, the compound components, or pharmaceutically acceptable salts thereof. Such kits will generally contain, in suitable container means, a pharmaceutically acceptable formulation. The kit may have a single container means, and/or it may have distinct container means for each compound.

When the components of the kit are provided in one and/or more liquid solutions, the liquid solution may be an aqueous solution, with a sterile aqueous solution being particularly preferred. The compound may also be formulated into a syringeable composition. In this case, the container means may itself be a syringe, pipette, and/or other such like apparatus, from which the formulation may be applied to an infected area of the body, injected into an animal, and/or even applied to and/or mixed with the other components of the kit.

The components of the kit may be provided as dried powder(s). When reagents and/or components are provided as a dry powder, the powder can be reconstituted by the addition of a suitable solvent. The solvent may be aqueous or organic. It is envisioned that the solvent may also be provided in another container means.

The container means will generally include at least one vial, test tube, flask, bottle, syringe and/or other container means. The kits may also comprise a second container means for containing a sterile, pharmaceutically acceptable buffer and/or other diluent.

Irrespective of the number and/or type of containers, the kits of the invention may also comprise, and/or be packaged with, an instrument for assisting with the injection/administration and/or placement of the ultimate pharmaceutical compound within the body of an animal. Such an instrument may be a syringe, pipette, forceps, and/or any such medically approved delivery vehicle, for example.

### N. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Multimodality Molecular Imaging of Glioblastoma Growth Inhibition with Vasculature-Targeting Fusion Toxin VEGF₁₂₁/rGEL

### Materials and Methods

⁶⁴Cu Labeling of DOTA-VEGF₁₂₁/rGel. The synthesis, expression, and purification of the fusion toxin VEGF₁₂₁/rGel were conducted as previously described (Veenendaal et al., 2002). 1,4,7,10-Tetraazacyclododedane-N,N′,N″,N′″-tetraacetic acid (DOTA) conjugation was performed as previously described with slight modifications and a reaction ratio of DOTA to VEGF₁₂₁/rGel of 100:1 (Cai et al., 2006a; Wu et al., 2005). DOTA-VEGF₁₂₁/rGel was purified by use of a PD-10 desalting column (GE Healthcare) and concentrated by use of a Centricon apparatus (Millipore). ⁶⁴CuCl₂ (74 MBq) was diluted in 300 μL of 0.1N sodium acetate buffer (pH 6.5) and then added to 50 μg of DOTA-VEGF₁₂₁/rGel. The reaction mixture was incubated for 1 h at 40° C. with constant shaking ⁶⁴Cu-DOTA-VEGF₁₂₁/rGel was then purified by use of a PD-10 column with phosphate-buffered saline (PBS) as the mobile phase. The average number of DOTA chelator molecules per VEGF₁₂₁/rGel molecule was determined as previously described (Cai et al., 2006a).

Cell-Binding Assay and Functional Assay. A cell-binding assay with VEGF₁₂₁/rGel and the DOTA-VEGF₁₂₁/rGel conjugate was performed as previously described with ¹²⁵I-VEGF₁₆₅ (specific activity, 74 TBq/mmol; GE Healthcare) as the radioligand (Cai et al., 2006b). The best-fit 50% inhibitory concentrations (IC₅₀s) for the PAE/KDR cells were calculated by fitting the data with GraphPad Prism (GraphPad Software, Inc.). Experiments were performed twice with triplicate samples.

Details of the functional assay were reported earlier (Cai et al., 2006b). The cell lysate was immunoblotted with an antiphosphotyrosine antibody (Santa Cruz Biotechnology) for phosphorylated KDR to compare the functional activities of DOTA-VEGF₁₂₁/rGel and VEGF₁₂₁/rGel. Tubulin was used as the loading control.

Orthotopic Glioblastoma Xenografts and Treatment Protocol. All animal procedures were performed according to a protocol approved by the Stanford University Administrative Panels on Laboratory Animal Care. Athymic nude mice (nu/nu; Harlan) at 4-6 wk of age were given intracranial injections in the right frontal lobe at coordinates 2 mm lateral and 0.5 mm anterior from the bregma and 2.5 mm intraparenchymally (Hsu et al., 2006). Each mouse was injected with 10⁵ firefly luciferase transfected U87MG human glioblastoma cells (U87MG-fLuc) suspended in 5 μL of PBS. Tumor cells were allowed to engraft for 7 d, at which point in vivo BLI and MRI were performed weekly to assess tumor growth. On day 34 after inoculation, BLI and MRI were conducted to assess tumor signal intensity and tumor volume before treatment. Mice were randomized to 2 groups: control (n=6) and VEGF₁₂₁/rGel treatment (n=9). A maximum tolerated dose (MTD) of 45 mg/kg for VEGF₁₂₁/rGel was previously established (Ran et al., 2005), and about 50% of the MTD was used for treatment purposes (120 μg×4=480 μg per mouse; 24 mg/kg administered every other day for a total of 4 doses). Saline administration was used as a single control on the basis of previous studies that demonstrated no impact on tumor growth when the same doses of rGel alone were administered (Mohamedali et al., 2005; Veenendaal et al., 2002). It was also believed that no therapeutic advantages would be gained by using free VEGF₁₂₁ as a control, as it would most likely induce, rather than inhibit, angiogenesis. Preliminary work in our laboratory has also shown no effects of rGel alone on subcutaneous U87MG glioblastoma growth.

Treatment with VEGF₁₂₁/rGel began on day 35 after inoculation, the time at which previous experiments showed that tumor angiogenesis and growth began to increase exponentially (Hsu et al., 2006). For treatment, the animals in the treatment group each received a 120-μL intraperitoneal injection of VEGF₁₂₁/rGel (1 mg/mL) every other day for a total of 4 treatments; the control animals each received equivalent injections of saline. The weights of all of the animals were recorded every other day to monitor potential toxicity effects. The animals were sacrificed after 4 doses of VEGF₁₂₁/rGel and perfused with 20 mL of cold saline and then 20 mL of 10% formalin. The brains were then embedded in paraffin for histologic analysis.

BLI. Intracranial tumor growth bioluminescence was assessed by use of a Xenogen IVIS 200 small-animal imaging system (Xenogen Corp.) (Hsu et al., 2006). For BLI, an average of ten 1-min-exposure bioluminescence acquisitions were collected between 0 and 40 min after substrate injection to confirm the peak photon emission recorded as the maximum photon efflux per second. Identical illumination settings (lamp voltage, filters, f-stop, field of view, binning, excitation filter block, and emission filter open) were used to acquire all images throughout the study. Data were analyzed by use of total photon flux emission (photons per second) in a region of interest covering the entire brain. BLI was conducted on a weekly basis after tumor inoculation and at 24 h after the administration of each VEGF₁₂₁/rGel dose to evaluate treatment progression.

MRI. Intracranial tumor growth was confirmed by gadolinium-enhanced MRI with a 4.7-T small-animal MRI system (Omega, GE Healthcare) equipped with a volume-based transmit/receive coil and an inner diameter of 4 cm (Hsu et al., 2006). Tumor volume was assessed by use of the free-hand region-of-interest function of Image J software (National Institutes of Health). MRI reconstruction of tumor volume has been shown to have a strong correlation (r=0.96) with traditional histologic reconstruction (Schmidt et al., 2004), with MRI having the disadvantage of partial-volume effects and the histologic technique having problems with tissue loss and shrinkage.

PET Imaging. The details of our PET image acquisition and quantification procedure were previously described (Chen et al., 2005; Xiong et al., 2006; Chen et al., 2006). Images were reconstructed by use of a 2-dimensional ordered-subsets expectation maximum algorithm with no attenuation or scatter correction (Visvikis et al., 2001). Mice were intravenously injected with 5-10 MBq of ⁶⁴Cu-DOTA-VEGF₁₂₁/rGel or ¹⁸F-FLT via the tail vein under 1%-2% isoflurane anesthesia. A blocking experiment was performed for ⁶⁴Cu-DOTA-VEGF₁₂₁/rGel by injecting 200 μg of VEGF₁₂₁ before injecting ⁶⁴ Cu-DOTA-VEGF₁₂₁/rGel. ¹⁸F-FLT was synthesized by reacting no-carrier-added ¹⁸F-fluoride with the precursor 5′-O-benzoyl-2,3′-anhydrothymidine and then performing hydrolysis with 1% NaOH in a TRACERlab FX_{FN} automatic synthesis module (GE Healthcare) (Tseng et al., 2005). PET scans (3-20 min, static) were obtained at various time points up to 48 h after injection with ⁶⁴Cu-DOTA-VEGF₁₂₁/rGel and at 1 and 2 h after injection with ¹⁸F-FLT.

Histologic Analysis. Paraffin sections (7 μm thick) were deparaffinized in xylene, rehydrated in graded alcohols, transferred to PBS, and then stained with hematoxylin and eosin (H&E) (Biogenex Laboratories). For Ki67 staining, an antigen retrieval kit with pepsin was used in accordance with the manufacturer's guidelines (Abcam). Endogenous peroxidase was blocked with 3% hydrogen peroxide in PBS for 12 min. Sections were washed 3 times with PBS and incubated for 20 min at room temperature with a protein-blocking solution containing PBS (pH 7.5), 5% normal horse serum, and 1% normal goat serum. Excess blocking solution was drained, and the samples were incubated for 18 h at 4° C. with a 1:100 dilution of Ki67 (Lab Vision). After being rinsed 4 times with PBS and incubated for 1 h at room temperature with the secondary antibody, the sections were rinsed with PBS and incubated for 5 min with diaminobenzidine (Research Genetics). Finally, the sections were attached to glass slides with mounting medium (Vectashield; Vector Labs), coverslips were added, and the sections were stored at −20° C.

Immunofluorescent Terminal Deoxynucleotidyltransferase-Mediated Biotin-dUTP Nick-End Labeling (TUNEL) Analysis. Paraffin sections were deparaffinized as described earlier. TUNEL analysis was performed by use of a commercial kit (Roche Applied Science) in accordance with the manufacturer's instructions. Samples were fixed with 10% formalin (methanol free) for 10 min at room temperature, washed with PBS, and permeabilized by incubation with 0.2% polyethylene glycol p-(1,1,3,3-tetramethylbutyl)-phenyl ether (Triton X-100; Sigma) in PBS (v/v) for 15 min. After the samples were incubated with equilibration buffer (from the kit), a reaction buffer containing equilibration buffer (45 mL), a nucleotide mixture (5 mL), and terminal deoxynucleotidyltransferase (1 mL) was added and incubated with the samples in a humidified chamber for 1 h at 37° C. in the dark. The reaction was terminated by immersing the samples in 30 mM NaCl and 3 mM sodium citrate (pH 7.2) for 15 min; this step was followed by 3 washes to remove unincorporated fluorescein-dUTP. The nuclei were stained with 4′,6′-diamidino-2-phenylindole (DAPI; 1 mg/mL) for 10 min.

Immunofluorescence Staining. Frozen tissue sections (7 μm thick) were fixed with cold acetone. Samples were washed 3 times with PBS and incubated for 20 min at room temperature with a protein-blocking solution containing PBS (pH 7.5), 5% normal horse serum, and 1% normal goat serum. Excess blocking solution was drained, and the samples were incubated for 18 h at 4° C. with a 1:100 dilution of rat monoclonal anti-CD31 antibody (BD PharMingen). Samples were rinsed 4 times with PBS and mounted with DAPI mounting medium for staining of the nuclei (Vectashield).

Fluorescence images were acquired by use of an Axiovert 200M fluorescence microscope (Carl Zeiss MicroImaging, Inc.) equipped with a DAPI (excitation 365 nm; emission 397 nm), Texas Red (excitation 535 nm; emission 610 nm), and Cy5.5 (excitation 665 nm; emission 725 nm) filter set. Images were obtained with a thermoelectrically cooled charge-coupled device (Micromax, model RTE/CCD-576; Princeton Instruments Inc.) and analyzed with MetaMorph software (version 6.2r4; Molecular Devices Corp.).

Data Processing and Statistics. All of the data presented are given as the mean±SD of n independent measurements. Statistical analysis was performed with a 1-way ANOVA for multiple groups and an unpaired Student t test; statistical significance was assigned for P values of <0.05 (GraphPad Prism).

### Results

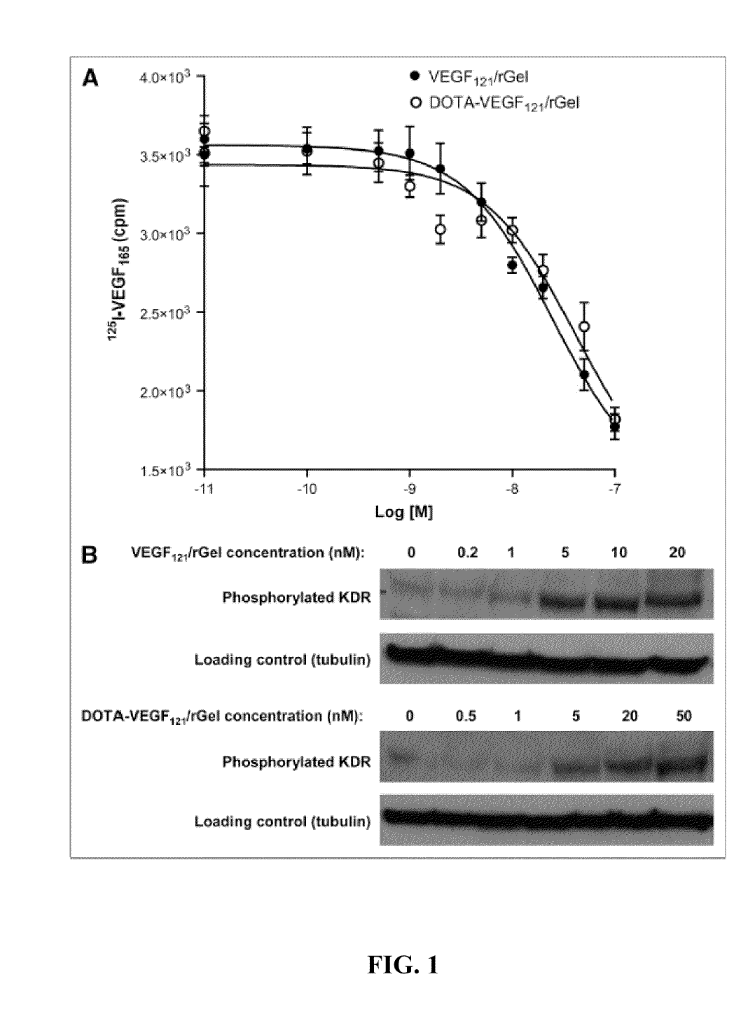

Binding and Functional Assays for DOTA-VEGF₁₂₁/rGel. VEGF₁₂₁/rGel and DOTA-VEGF₁₂₁/rGel were able to inhibit ¹²⁵I-VEGF₁₆₅ binding to VEGFR-2 expressed on PAE/KDR cells in a dose-dependent manner (FIG. 1A). IC₅₀ values of 24.5 and 40.6 nM were obtained for VEGF₁₂₁/rGel and DOTA-VEGF₁₂₁/rGel, respectively, indicating that no significant change in VEGF₁₂₁/rGel binding affinity was caused by DOTA conjugation. Western blot analysis (functional assay) of VEGF₁₂₁/rGel and DOTA-VEGF₁₂₁/rGel with PAE/KDR cells revealed a slight decrease in the level of expression of phosphorylated KDR after DOTA conjugation (FIG. 1B). Increased levels of expression of phosphorylated KDR were observed at VEGF₁₂₁/rGel and DOTA-VEGF₁₂₁/rGel concentrations of ≧5 nM, with a consistent single protein band at 200 kDa.

⁶⁴Cu-DOTA-VEGF₁₂₁/rGel Tumor Accumulation. ⁶⁴Cu labeling of DOTA-VEGF₁₂₁/rGel, including final purification, took 90±10 min (n=3), and the radiolabeling yield was 85.2%±9.2% (on the basis of 37 MBq of ⁶⁴Cu per 25 μg of DOTA-VEGF₁₂₁/rGel; n=3). The specific activity of ⁶⁴Cu-DOTA-VEGF₁₂₁/rGel was 1.3±0.1 GBq/mg, and the radiochemical purity was greater than 98%. The number of DOTA molecules per VEGF₁₂₁/rGel molecule was found to be 3.3±0.1 (n=4).

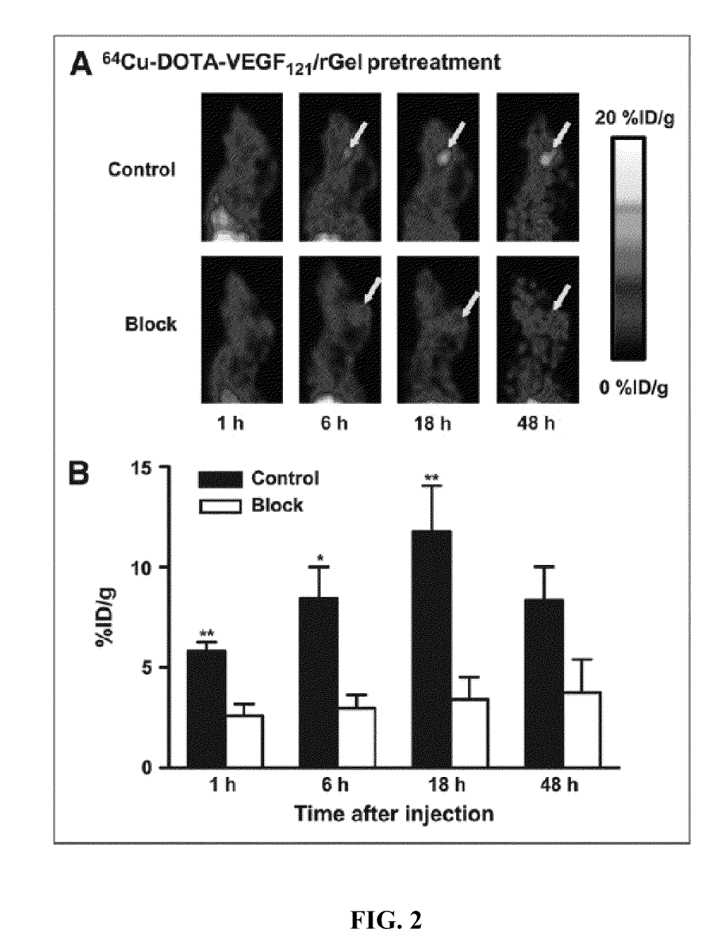

⁶⁴Cu-DOTA-VEGF₁₂₁/rGel exhibited high pretreatment tumor accumulation and retention and high tumor-to-background contrast from 1 to 48 h after injection in glioblastoma xenografts (FIG. 2A). Tumor accumulation at 1 h after injection was 5.8±0.5 percentage injected dose per gram (% ID/g) (n=3) and steadily increased and peaked at about 18 h after injection (11.8±2.3% ID/g). At 46 h after injection, tumor uptake decreased to 8.4±1.7% ID/g. Although the tumor uptake of ⁶⁴Cu-DOTA-VEGF₁₂₁/rGel remained high as tumor sizes increased, there was no clear relationship between tumor size and tracer uptake. ⁶⁴Cu-DOTA-VEGF₁₂₁/rGel was cleared through both the hepatic and the renal pathways (Jekunin et al., 1996). A blocking experiment with 200 μg of VEGF₁₂₁ injected before ⁶⁴Cu-DOTA-VEGF₁₂₁/rGel injection revealed a significant reduction in ⁶⁴Cu-DOTA-VEGF₁₂₁/rGel uptake (FIG. 2B). PET clearly demonstrated VEGFR-specific tumor uptake of ⁶⁴Cu-DOTA-VEGF₁₂₁/rGel, a finding that provided the basis for the following treatment protocol. On the basis of the in vivo pharmacokinetics of ⁶⁴Cu-DOTA-VEGF₁₂₁/rGel, VEGF₁₂₁/rGel was administered every other day for the VEGFR-2-targeted treatment of orthotopic U87MG glioblastomas.

BLI and MRI Monitoring of Glioblastoma Growth Inhibition. Before treatment with VEGF₁₂₁/rGel, we first validated the use of BLI for the assessment of longitudinal tumor growth in comparison with gadolinium-enhanced MRI. There was a significant increase in both BLI and MRI tumor signal intensities as tumors grew from day 21 to day 46 after inoculation, and logarithmic transformation of tumor volumes assessed by BLI and MRI on the same day showed a strong linear correlation (r=0.89, n=14). The MRI tumor volume spanned 1-74 mm³, and increases in tumor volume had no significant effect on the time required to reach the peak BLI tumor signal intensity from day 1 to day 46 after inoculation.

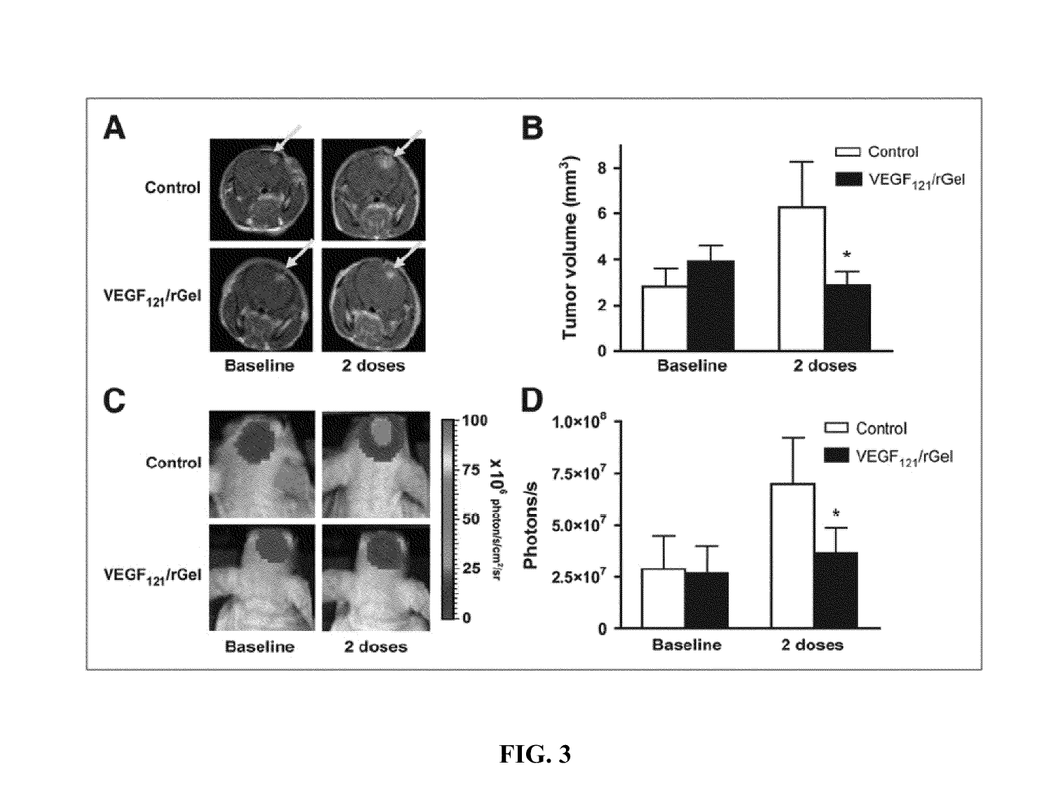

There were no significant differences in MRI tumor volume or BLI tumor signal intensity between the control group and the VEGF₁₂₁/rGel treatment group on day 34 after inoculation. Control and treatment group tumor volumes (as determined by MRI) at baseline were 2.8±0.8 and 3.9±0.7 mm³, respectively (FIGS. 3A and 3B). Peak BLI tumor signal intensities in the control and treatment groups were 2.9×10⁷±1.6×10⁷ and 2.7×10⁷±1.3×10⁷ photons per second, respectively (FIGS. 3C and 3D). There were significant decreases in MRI tumor volume and BLI tumor signal intensities in the treatment group compared with the control group after only 2 of the 4 total doses of VEGF₁₂₁/rGel. The MRI tumor volume in the control group increased to 6.3±2.0 mm³, whereas that in the treatment group decreased to 2.9±0.6 mm³ (P<0.05). Similarly, the BLI tumor signal intensity in the control group increased to 7.0×10⁷±2.3×10⁷ photons per second, whereas that in the treatment group increased only slightly, to 3.7×10⁷±1.2×10⁷ photons per second (P<0.05).

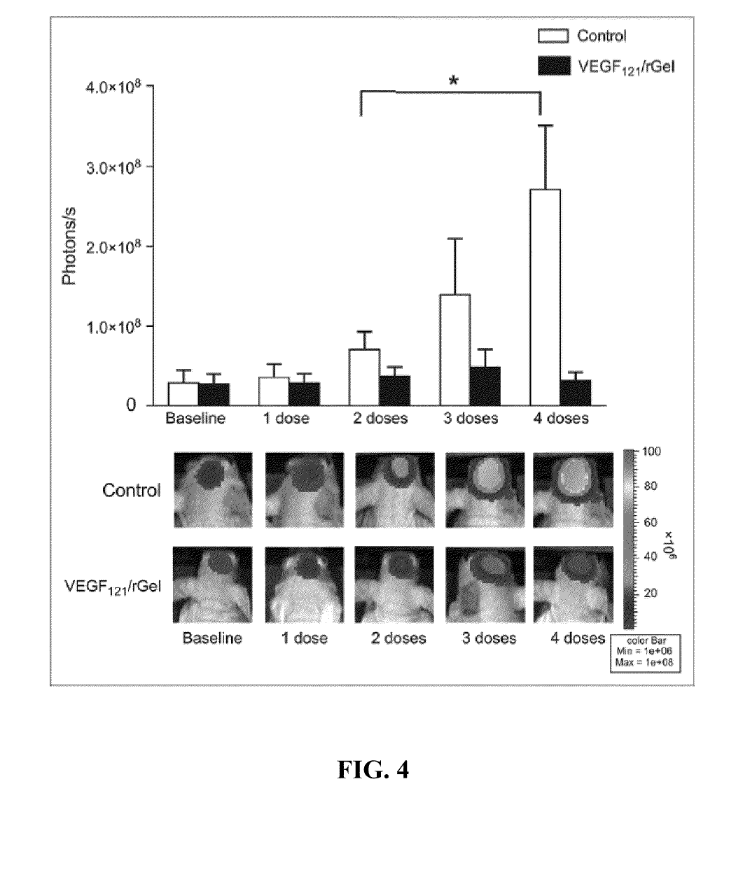

The difference in MRI tumor size between the 2 groups reached a peak after 4 doses of VEGF₁₂₁/rGel, with a significant 8.7-fold-lower BLI tumor signal intensity in the treatment group than in the control group (P<0.05) (FIG. 4). There was no difference in starting body weight between the 2 groups (control: 23.2±0.5 g; treatment: 21.6±0.5 g), but there was a significant difference in body weight loss between the 2 groups after therapy completion (control: 3.7±0.3 g; treatment: 5.8±0.7 g), possibly indicating some normal tissue cytotoxic side effects.

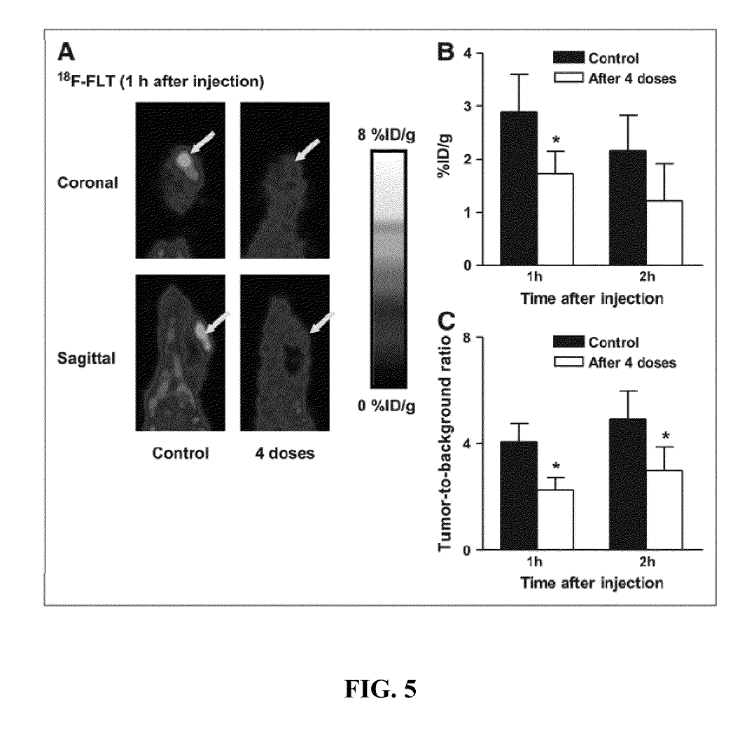

¹⁸F-FLT PET Imaging of VEGF₁₂₁/rGel Treatment. ¹⁸F-FLT PET revealed significant differences in tumor uptake and the ratio of tumor uptake to background uptake (T/B ratio) between the control group and the treatment group (FIG. 5A), with no significant differences between 1 h and 2 h after injection. ¹⁸F-FLT tumor accumulation values at 1 h after injection (n=3) were 2.9±0.7% ID/g in the control group and 1.7±0.4% ID/g after 4 doses of VEGF₁₂₁/rGel (P<0.05), indicating decreased DNA synthesis during treatment (FIG. 5B). Showing a trend similar to that for tumor uptake, the T/B ratios at 1 h after injection (n=3) were 4.1±0.7 and 2.3±0.5 for the control and treatment groups, respectively (P<0.05) (FIG. 5C). Both tumor uptake and the T/B ratio at 2 h after injection showed the same trends as those observed at 1 h after injection.

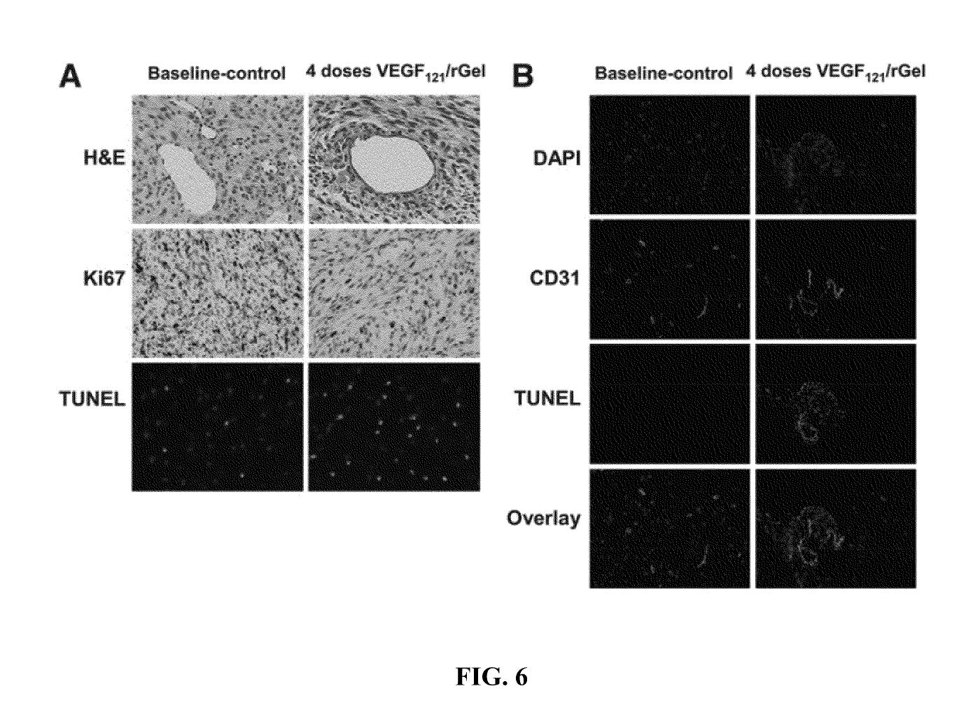

Histologic Analysis of VEGF₁₂₁/rGel Glioblastoma Inhibition. Histologic analysis of paraffin-embedded tumor sections stained with H&E, Ki67, and TUNEL revealed significant morphologic, proliferative, and apoptotic differences between the control group and the treatment group (FIG. 6A). In VEGF₁₂₁/rGel-treated mice, H&E analysis revealed marked degradation and weakening of tumor neovasculature. In contrast, control mice showed healthy, mature tumor endothelium. Ki67 analysis revealed a 5±2% proliferative index in the treated mice, whereas the control mice had a proliferative index of 17±4%. Quantitative TUNEL-positive cell analysis revealed increased DNA fragmentation and apoptosis in the treated mice compared with the control mice. Tumors in the control mice had 4.1±0.9% TUNEL-positive cells, whereas those in the treated mice had 12.8±2.2% TUNEL-positive cells. Immunohistochemical analysis with DAPI, CD31, and TUNEL staining revealed increased apoptosis on tumor vasculature and surrounding cells in the treated mice compared with the control mice, indicating specific VEGF₁₂₁/rGel-induced tumor vasculature damage (FIG. 6B). These ex vivo results indicate that VEGF₁₂₁/rGel inhibits glioblastoma growth through tumor vasculature degradation with a subsequent decrease in DNA synthesis and increase in tumor cell apoptosis.

All of the methods disclosed and claimed herein can be made and executed without undue experimentation in light of the present disclosure. While the methods of this invention have been described in terms of preferred embodiments, it will be apparent to those of skill in the art that variations may be applied to the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,554,101
U.S. Pat. No. 4,677,064
U.S. Pat. No. 4,680,338
U.S. Pat. No. 5,141,648
U.S. Pat. No. 5,247,070
U.S. Pat. No. 5,422,104
U.S. Pat. No. 5,519,119
U.S. Pat. No. 5,563,250
U.S. Pat. No. 5,606,023
U.S. Pat. No. 5,652,353
U.S. Pat. No. 5,773,582
U.S. Pat. No. 5,856,456
U.S. Pat. No. 5,880,270
U.S. Patent Publn. 20020114783
U.S. Patent Publn. 20030082159
U.S. Ser. No. 10/074,596
Adams and Schier, J. Immunol. Methods, 231:249-260, 1999.
Akiyama et al., Mol. Pharmacol., 68:1543-1550, 2005.
Alberico et al., Surg. Oncol. Clin. N. Am., 13(1):13-35, 2004.
Anand-Apte et al., Biochem. Cell. Biol., 74:853-862, 1996.
Antibodies: A Laboratory Manual, Cold Spring Harbor Laboratory, 1988.
Atwell et al., Protein Eng., 12:597-604, 1999.
Bergers et al., Science, 284:808-812, 1999.
Bigg et al., J. Biol. Chem., 272:15496-15500, 1997.
Bikfalvi A, Bicknell, Trends Pharmacol Sci., 23:576-582, 2002.
Bird et al., Science, 242:423-426, 1988.
Bittner et al., Methods in Enzymol, 153:516-544, 1987.
Bodanszky et al., J. Antibiot., 29(5):549-53, 1976.
Boehm et al., Nature, 390:404-407, 1997.
Brekken et al., Cancer Res., 60:5117-5124, 2000.
Brooks et al., Cell, 79:1157-1164, 1994.
Burbage et al., Leuk Res., 21(7):681-690, 1997.
Cai et al., J. Nucl. Med., 47:2048-2056, 2006.
Cai et al., Cancer Res., 66:9673-9681, 2006.
Cao et al., J. Biol. Chem., 272: 22924-22928, 1997.
Cao et al., Gene Ther., 5(8):1130-1136, 1998.
Caruthers et al., Nucleic Acids Symp. Ser., (7):215-223, 1980.
Chaudhary et al., Proc. Natl. Acad. Sci. USA, 87(3):1066-1070, 1990.
Chen et al., Mol. Imaging Biol., 8:9-15, 2006.
Chen et al., Neoplasia., 7:271-279, 2005.
Chow and Kempe, Nucleic Acids Res., 9(12):2807-2817, 1981.
Colcher et al., J. Natl. Cancer Inst., 82:1191-1197, 1990.
Colcher et al., J. Nucl. Med., 42:225-241, 1998.
Crea and Horn, Nucleic Acids Res., 8(10):2331-2348, 1980.
Creighton, In: Proteins Structures And Molecular Principles, W.H. Freeman and Co., N.Y. 50-60, 1983.
Culver et al., Science, 256(5063):1550-1552, 1992.
Eggert et al., Clin. Cancer Res., 6:1900-1908, 2000.
Fabbro et al., Anticancer Drug Des., 15:17-28, 2000.
Fine et al., Gynecol. Oncol., 76:33-39, 2000.
Fischer et al., Brain Pathol., 15:297-310, 2005.
Furumatsu et al., Int. J. Cancer, 97:313-322, 2002.
Gomez et al., Eur. J. Cell. Biol., 74: 111-122, 1997.
Greene et al., J. Biol. Chem., 271:30375-30380, 1996.
Henson et al., AJNR Am. J. Neuroradiol., 25(6):969-972, 2004.
Hsu et al., Mol. Imaging Biol., 8:315-323, 2006.
Huston et al., Biochemistry, 27(25):8945-8952, 1988.
Itakura et al., Int. J. Cancer, 85:27-34, 2000.
Jekunen et al., Acta Oncol., 35:267-271, 1996.
Ji et al., Biochem. Biophys. Res. Commun., 247:414-419, 1998.
Kantor et al., Hybridoma., 1:473-482, 1982.
Kirsch et al., Cancer Res., 58:4654-4659, 1998.
Kumamoto et al., J. Oral. Pathol. Med., 31:28-34, 2002.
Kuroki et al., Anticancer Res., 20(6A):4067-4071 2000.
Kyte and Doolittle, J. Mol. Biol., 157(1):105-132, 1982.
Lidor et al., Am. J. Obstet. Gynecol., 177(3):579-585, 1997.
Liu et al., Arch. Biochem. Biophys., 424:33-43, 2004.
Liu et al., Am. J. Physiol., 284:C1073-1082, 2003.
Luster and Leder, J. Exp. Med., 178:1057-1065, 1993.
Macey et al., Am. J. Physiol. Imaging, 3:1-6, 1988.
Martin et al., Oncogene, 13:569-576, 1996.
Massuda et al., Proc. Natl. Acad. Sci. USA, 94(26):14701-14706, 1997.
Mauceri et al., Nature, 394:287-291, 1998.
Mohamedali et al., Neoplasia, 7:912-920, 2005.
Mujoo et al., Cancer Immunol. Immunother., 40:339-345, 1995.
Musso et al., J. Hepatol., 26:593-605, 1997.
Nechushtan et al., J. Biol. Chem., 272(17):11597-11603, 1997.
O′Reilly et al., Cell, 88:277-85, 1997.
Price et al., Cell Growth Differ., 12:129-135, 2001.
Ran et al., Neoplasia., 7:486-496, 2005.
Rasey et al., J. Nucl. Med., 43:1210-1217, 2002.
Remington Pharmaceutical Sciences, 18th Ed. Mack Printing Company, 1289-1329, 1990.
Rosenblum et al., Cancer Commun., 3:21-27, 1991.
Rosenblum et al., Cancer Immunol. Immunother., 39:397-400, 1994.
Rosenblum et al., Cancer Immunol. Immunother., 40:322-328, 1995.
Rosenblum et al., Cancer Immunol. Immunother., 42:115-121, 1996.
Rosenblum et al., Cancer Res., 63:3995-4002, 2003.
Rosenblum et al., Clin. Cancer Res., 5:865-874, 1999.
Saha et al., Am. J. Physiol., 267(1 Pt 1):E95-101, 1994.
Sambrook and Russell, Molecular Cloning: A Laboratory Manual 3^{rd} Ed., Cold Spring Harbor Laboratory Press, 2001.
Sasaki et al., J. Mol. Biol., 301(5):1179-1190, 2000.
Schmidt et al., J. Neurooncol., 68:207-215, 2004.
Senger et al., Cancer Metastasis Rev., 12:303-324, 1993.
Sgadari et al., Blood, 89:2635-2643, 1997.
Sgadari et al., Proc. Natl. Acad. Sci. USA, 93:13791-13796, 1996.
Stetler-Stevenson et al., J. Biol. Chem., 265:13933-13938, 1990.
Strunk and Schild, Eur. Radiol., 14(6):1055-1062, 2004.
Tanaka et al., Genes Cells, 3:29-37, 1998.
Tannenbaum et al., J. Immunol., 161:927-932, 1998.
Barclay et al., In: The Leucocyte Antigen Facts Book, Academic Press, 1993.
Thompson, In: The Cytokine Handbook, Academic Press, San Diego, 1994.
Tseng et al., J. Nucl. Med., 46:1851-1857, 2005.
Valente et al., Int. J. Cancer, 75:246-253, 1998.
Veenendaal et al., Proc. Natl. Acad. Sci. USA, 99:7866-7871, 2002.
Visvikis et al., Eur. J. Nucl. Med., 28:1326-1335, 2001.
Wang et al., Biochemistry, 36:15026-15034, 1997.
Wawrzynczak and Thorpe, FEBS Lett., 207(2):213-216, 1986.
PCT Appln. WO 97/19179
Wu et al., J. Nucl. Med., 46:1707-1718, 2005.
Xiong et al., J. Nucl. Med., 47:130-139, 2006.
Zetter, Annu. Rev. Med., 49:407-424, 1998.

1. A pharmaceutical compound comprising an anti-cell proliferation moiety and a cell-specific targeting moiety, wherein the anti-cell proliferation moiety and cell-specific targeting moiety are directly bound to one another or bound to one another by a linker, and one or more chelator moieties are bound to the anti-cell proliferation moiety and/or the cell-specific targeting moiety.

2. The pharmaceutical compound of claim 1, wherein the cell-specific targeting moiety is further defined as an antibody, a growth factor, a hormone, a polypeptide, a peptide, an aptamer, or a cytokine.

3. (canceled)

4. The pharmaceutical compound of claim 2, wherein the antibody is selected from the group consisting of a full-length antibody, chimeric antibody, Fab′, Fab, F(ab′)₂, single domain antibody (DAB), Fv, single chain Fv (scFv), minibody, diabody, triabody, or a mixture thereof.

5. (canceled)

6. The pharmaceutical compound of claim 1, wherein the cell-specific targeting moiety is a vascular endothelial cell-specific targeting moiety.

7. The pharmaceutical compound of claim 6, wherein the vascular endothelial cell-specific targeting moiety is VEGF, FGF, integrin, fibronectin, I-CAM, or PDGF.

8. (canceled)

9. The pharmaceutical compound of claim 7, wherein the vascular endothelial-specific targeting moiety is a VEGF is an isoform that is VEGF₁₂₁, VEGF₁₆₅, VEGF₁₈₉, or VEGF₂₀₆.

10. The pharmaceutical compound of claim 9, wherein the isoform is VEGF₁₂₁.

11. The pharmaceutical compound of claim 10, wherein the VEGF sequence comprises SEQ ID NO:2, SEQ ID NO:3, SEQ ID NO:4, SEQ ID NO:5, SEQ ID NO:6, SEQ ID NO:7, or SEQ ID NO:8.

12. The pharmaceutical compound of claim 2, wherein the cell-specific targeting moiety is a growth factor.

13. The pharmaceutical compound of claim 2, wherein the cell-specific targeting moiety is a growth factor that is transforming growth factor, epidermal growth factor, insulin-like growth factor, fibroblast growth factor, heregulin, platelet-derived growth factor, vascular endothelial growth factor, or hypoxia inducible factor.

14. (canceled)

15. The pharmaceutical compound of claim 2, wherein the cell-specific targeting moiety is a hormone that is human chorionic gonadotropin, gonadotropin releasing hormone, an androgen, an estrogen, thyroid-stimulating hormone, follicle-stimulating hormone, luteinizing hormone, prolactin, growth hormone, adrenocorticotropic hormone, antidiuretic hormone, oxytocin, thyrotropin-releasing hormone, growth hormone releasing hormone, corticotropin-releasing hormone, somatostatin, dopamine, melatonin, thyroxine, calcitonin, parathyroid hormone, glucocorticoids, mineralocorticoids, adrenaline, noradrenaline, progesterone, insulin, glucagon, amylin, erythropoitin, calcitriol, calciferol, atrial-natriuretic peptide, gastrin, secretin, cholecystokinin, neuropeptide Y, ghrelin, PYY.sub.3-36, insulin-like growth factor-1, leptin, thrombopoietin, angiotensinogen, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, or IL-36.

16. The pharmaceutical compound of claim 2, wherein the cell-specific targeting moiety is a cytokine.

17. The pharmaceutical compound of claim 16, wherein the cell-specific targeting moiety is a cytokine that is IL1, IL2, IL3, IL4, IL5, IL6, IL7, IL8, IL9, IL10, IL11, IL12, IL13, IL14, IL15, IL-16, IL-17, IL-18, granulocyte-colony stimulating factor, macrophage-colony stimulating factor, granulocyte-macrophage colony stimulating factor, leukemia inhibitory factor, erythropoietin, granulocyte macrophage colony stimulating factor, oncostatin M, leukemia inhibitory factor, interferon-γ, interferon-α, interferon-β, LT-β, CD40 ligand, Fas ligand, CD27 ligand, CD30 ligand, 4-1BBL, TGFβ, IL 1α, IL-1β, IL-1 RA, MIF, or IGIF.

18. The pharmaceutical compound of claim 1, wherein the anti-cell proliferation moiety is further defined as an apoptosis-inducing moiety.

19. The pharmaceutical compound of claim 18, wherein the apoptosis-inducing moiety is a granzyme, a Bcl-2 family member, cytochrome C, or a caspase.

20. The pharmaceutical compound of claim 19, wherein the apoptosis-inducing moiety is a granzyme that is granzyme A, granzyme B, granzyme C, granzyme D, granzyme E, granzyme F, granzyme G, granzyme H, granzyme I, granzyme J, granzyme K, granzyme L, granzyme M, or granzyme N.

21. The pharmaceutical compound of claim 18, wherein the apoptosis-inducing moiety is a Bcl-2 family member that is Bax, Bak, Bcl-Xs, Bad, Bid, Bik, Hrk, and Bok.

22. The pharmaceutical compound of claim 18, wherein the apoptosis-inducing moiety is a caspase that is caspase-1, caspase-2 caspase-3, caspase-4, caspase-5, caspase-6, caspase-7, caspase-8, caspase-9, caspase-10, caspase-11, caspase-12, caspase-13, or caspase-14.

23. The pharmaceutical compound of claim 1, wherein the anti-cell proliferation moiety is further defined as a cytotoxic agent.

24. (canceled)

25. The pharmaceutical compound of claim 23, wherein the cytotoxic agent is a recombinant ribosome-inhibiting protein (RIP).

26. The pharmaceutical compound of claim 25, wherein the ribosome-inhibiting protein (RIP) is gelonin, maize RIP, saporin, ricin, ricin A chain, barley RIP, momordin, alpha-momorcharin, beta-momorcharin, Shiga-like RIPE, or a-sarcin.

27. The pharmaceutical compound of claim 26, wherein the ribosome-inhibiting protein is gelonin.

28. The pharmaceutical compound of claim 23, wherein the cytotoxic agent is TNF-α, Prodigiosin, Pseudomonas exotoxin, Clostridium difficile Toxin B, Helicobacter pylori VacA, Yersinia enterocolitica YopT, Violacein, diethylenetriaminepentaacetic acid, irofulven, Diptheria toxin, mitogillin, ricin, botulinum toxin, cholera toxin, saporin 6, abrin, trichosanthin, dodecandrin, tricokirin, bryodin, or luffin.

29. (canceled)

30. The pharmaceutical compound of claim 1, wherein the cell-specific targeting moiety and the anti-cell proliferation moiety are chemically conjugated.

31. The pharmaceutical compound of claim 1, wherein the cell-specific targeting moiety and the anti-cell proliferation moiety are comprised in a fusion polypeptide.

32. The pharmaceutical compound of claim 1, wherein the pharmaceutical compound is of formula:
R₁-(L₁)n₁-R₂-(L₂)n₂-R₃, (I)
R₃-(L₁)n₁-R₁-(L₂)n₂-R₂, (II)
R₂-(L₁)n₁-R₁-(L₂)n₂-R₃, or (III)
R₃-(L₁)n₁-R₂-(L₂)n₂-R₁, (IV)
wherein R₁ is an anti-cell proliferation moiety, R₂ is a cell-specific targeting moiety, R₃ is a chelator moiety, n₁ and n₂ are independent 0 or 1, L₁ is a first linker, and L₂ is a second linker.

33. The pharmaceutical compound of claim 32, wherein n₁ or n₂ is zero.

34. The pharmaceutical compound of claim 1, wherein n₁ and n₂ are zero.

35. The pharmaceutical compound of claim 32, wherein L₁ or L₂ is G₄S, (G₄S)₂, (G₄S)₃, 218 linker, an enzymatically cleavable linker, or a pH cleavable linker.

36. The pharmaceutical compound of claim 35, wherein the linker is G₄S.

37. The pharmaceutical compound of claim 1, wherein the compound comprises a chelator moiety that is selected from the group consisting of DOTA, DTPA, DMSA, EDTA, Cy-EDTA, EDTMP, DTPA, CyDTPA, Cy2DTPA, BOPTA, DTPA-MA, DTPA-BA, DTPMP, TRITA, TETA, DOTMA, DOTA-MA, HP-DO3A, pNB-DOTA, DOTP, DOTMP, DOTEP, DOTPP, DOTBzP, DOTPME, HEDP, DTTP, an N₃S triamidethiol (MAG3), DADS, MAMA, DADT, a diaminetetrathiol, an N₂P₂ dithiol-bisphosphine, a 6-hydrazinonicotinic acid, a propylene amine oxime, a tetraamine, a cyclal, and a cyclam.

38. The pharmaceutical compound of claim 37, wherein the compound comprises a chelator moiety that is DOTA or SarAr.

39. The pharmaceutical compound of claim 1, further comprising a valent metal ion attached to a chelator moiety.

40. The pharmaceutical compound of claim 39, wherein the valent metal ion is Cu-64, Cu-60, Cu-61, Cu-62, Cu-67, Lu-177, Zr-89, Y-86, Tc-99m, In-111, T1-201, Ga-67, Ga-68, As-72, Re-186, Re-188, Ho-166, Y-90, Sm-153, Sr-89, Gd-157, Bi-212, and Bi-213.

41. (canceled)

42. The pharmaceutical compound of claim 1, wherein the cell-specific targeting moiety is VEGF₁₂₁, the anti-cell proliferation moiety is recombinant gelonin, and a chelator moiety is DOTA.

43. The pharmaceutical compound of claim 42, wherein Cu-64 is chelated to a chelator moiety.

44. The pharmaceutical compound of claim 1, wherein the cell-specific targeting moiety is scFvMEL, the anti-cell proliferation moiety is TNF-α, and a chelator moiety is DOTA.

45. The pharmaceutical compound of claim 44, wherein Cu-64 is chelated to a chelator moiety.

46. The pharmaceutical compound of claim 1, wherein the cell-specific targeting moiety is scFv23, the anti-cell proliferation moiety is TNF-α, and the chelator moiety is DOTA.

47. The pharmaceutical compound of claim 46, wherein Cu-64 is chelated to a chelator moiety.

48. A pharmaceutical compound of formula:
R₁-(L₁)n₁-R₃-(L₂)n₂-R₂ or (V)
R₂-(L₁)n₁-R₃-(L₂)n₂-R₁, (VI)
wherein R₁ is an anti-cell proliferation moiety that is gelonin, maize RIP, saporin, ricin, ricin A chain, barley RIP, momordin, alpha-momorcharin, beta-momorcharin, Shiga-like RIPE, a-sarcin, TNF-α, Prodigiosin, Pseudomonas exotoxin, Clostridium difficile Toxin B, Helicobacter pylori VacA, Yersinia enterocolitica YopT, Violacein, diethylenetriaminepentaacetic acid, irofulven, Diptheria toxin, mitogillin, ricin, botulinum toxin, cholera toxin, saporin 6, abrin, trichosanthin, dodecandrin, tricokirin, bryodin, or luffin; R₂ is a cell-specific targeting moiety; R₃ is a chelator moiety; nᵢ and n₂ are independently 0 or 1; L₁ is a first linker; and L₂ is a second linker.

49. The compound of claim 47, wherein the anti-cell proliferation moiety is gelonin or TNF-α.

50. (canceled)

51. A method for treating a hyperproliferative disease in a subject, comprising administering to the subject a pharmaceutically effective amount of a compound as set forth in claim 1.

52. (canceled)

53. The method of claim 51, wherein the mammal is a human.

54. The method of claim 51, wherein the hyperproliferative disease is a disease associated with neovascularization.

55. The method of claim 54, wherein the disease associated with neovascularization is a cancer.

56. The method of claim 55, wherein the cancer is brain cancer, breast cancer, lung cancer, prostate cancer, ovarian cancer, liver cancer, cervical cancer, colon cancer, renal cancer, skin cancer, head and neck cancer, bone cancer, esophageal cancer, bladder cancer, uterine cancer, lymphatic cancer, stomach cancer, pancreatic cancer, testicular cancer, lymphoma, or leukemia.

57. The method of claim 56, wherein the cancer is brain cancer.

58. The method of claim 57, wherein the brain cancer is glioblastoma multiforme.

59. The method of claim 51, further comprising treating the subject with an additional anti-hyperproliferative disease therapy.

60. The method of claim 59, wherein the additional therapy is chemotherapy, surgery, radiation therapy, gene therapy, hormone therapy, or immunotherapy.

61-63. (canceled)

64. The method of claim 51, further comprising imaging the subject using a noninvasive technique, wherein the chelator moiety is chelated to a valent metal ion and the pharmaceutical compound is detectable in vivo using the non-invasive imaging technique.

65. The method of claim 64, wherein the imaging comprises MRI, MR spectroscopy, radiography, CT, ultrasound, planar gamma camera imaging, SPECT, or PET.

66-67. (canceled)

68. A method of diagnosing the presence of a hyperproliferative disease in a human subject, comprising:
(a) administering to a human subject a pharmaceutically effective amount of a compound of claim 1, wherein the chelator moiety is chelated to a valent metal; and
(b) performing a noninvasive imaging technique, wherein detection of an image identifies the subject as having a hyperproliferative disease.

69. (canceled)

70. The method of claim 68, wherein the hyperproliferative disease is cancer.

71. The method of claim 70, wherein the cancer is brain cancer, breast cancer, lung cancer, prostate cancer, ovarian cancer, liver cancer, cervical cancer, colon cancer, renal cancer, skin cancer, head and neck cancer, bone cancer, esophageal cancer, bladder cancer, uterine cancer, lymphatic cancer, stomach cancer, pancreatic cancer, testicular cancer, lymphoma, or leukemia.

72. The method of claim 71, wherein the cancer is brain cancer.

73. The method of claim 72, wherein the brain cancer is glioblastoma multiforme.

74. The method of claim 68, wherein the imaging comprises MRI, MR spectroscopy, radiography, CT, ultrasound, planar gamma camera imaging, SPECT, or PET.

75. A method for detecting a therapeutic response following treatment a patient with a hyperproliferative disease, comprising the steps of:
(a) administering to a subject with a hyperproliferative disease a pharmaceutically effective amount of a compound as set forth in claim 1, wherein the chelator moiety is chelated to a valent metal ion and is detectable in vivo using a noninvasive imaging technique;
(b) imaging the subject using the noninvasive imaging technique to obtain a first image;
(c) repeating step (a); and
(d) imaging the subject using the noninvasive imaging technique to obtain a second image,
wherein reduction of the size or intensity of the second image compared to the first image indicates presence of a therapeutic response.

76. (canceled)

77. The method of claim 75, wherein the hyperproliferative disease is cancer.

78. The method of claim 77, wherein the cancer is brain cancer, breast cancer, lung cancer, prostate cancer, ovarian cancer, liver cancer, cervical cancer, colon cancer, renal cancer, skin cancer, head and neck cancer, bone cancer, esophageal cancer, bladder cancer, uterine cancer, lymphatic cancer, stomach cancer, pancreatic cancer, testicular cancer, lymphoma, or leukemia.

79. The method of claim 78, wherein the cancer is brain cancer.

80. The method of claim 79, wherein the brain cancer is glioblastoma multiforme.

81. The method of claim 75, wherein the imaging comprises MRI, MR spectroscopy, radiography, CT, ultrasound, planar gamma camera imaging, SPECT, or PET.

82. (canceled)

83. A method for identifying a subject with a tumor that will respond to treatment with a pharmaceutical compound comprising a cell-specific targeting moiety conjugated to an anti-cell proliferation moiety, comprising the steps of:
(a) administering to a subject with a tumor a pharmaceutically effective amount of a pharmaceutical compound as set forth in claim 1, wherein the chelator moiety is chelated to a valent metal ion; and
(b) performing a noninvasive imaging technique on the subject;
wherein presence of a detectable image identifies the subject as having a tumor that will respond to treatment with the compound.

84. The method of claim 83, wherein the pharmaceutical compound is (64)Cu-DOTA-VEGF(121)/rGel.

85. The method of claim 83, wherein the tumor is a brain cancer, a breast cancer, a lung cancer, a prostate cancer, an ovarian cancer, a liver cancer, a cervical cancer, a colon cancer, a renal cancer, a skin cancer, a head and neck cancer, a bone cancer, an esophageal cancer, a bladder cancer, a uterine cancer, a lymphatic cancer, a stomach cancer, a pancreatic cancer, a testicular cancer, a lymphoma, or a leukemia.

86. The method of claim 85, wherein the tumor is a brain tumor.

87. The method of claim 86, wherein the brain tumor is glioblastoma multiforme.

88. The method of claim 83, wherein the imaging is MRI, MR spectroscopy, radiography, CT, ultrasound, planar gamma camera imaging, SPECT, or PET.

89. The method of claim 88, wherein the imaging is PET with (18)F-FLT.


SEQUENCE LISTING (11 sequences)

SEQ ID NO: 1 - protein, 14 aa, Artificial
  Synthetic peptide
EGKSSGSGSESKVD

SEQ ID NO: 2 - protein, 121 aa, Homo sapiens
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENCDKPRR

SEQ ID NO: 3 - protein, 206 aa, Homo sapiens
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 4 - protein, 189 aa, Homo sapiens
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 5 - protein, 183 aa, Homo sapiens
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRPCGPCSERRKHLFVQDPQTCKCSCKNTDSRCKARQLELNERTCRCDKPRR

SEQ ID NO: 6 - protein, 165 aa, Homo sapiens
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 7 - protein, 148 aa, Homo sapiens
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKM

SEQ ID NO: 8 - protein, 145 aa, Homo sapiens
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVCDKPRR

SEQ ID NO: 9 - protein, 5 aa, Artificial Sequence
  Synthetic peptide
GGGGS

SEQ ID NO: 10 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 11 - protein, 18 aa, Artificial Sequence
  Synthetic peptide
KESGSVSSEQLAQFRSLD